(19) United States
(12) Patent Application Publication
(10) Pub. No.: US 2025/0041052 A1
(43) Pub. Date: Feb. 6, 2025

(54) CRIMPER SYSTEMS, DEVICES, AND METHODS OF USING THE SAME

(71) Applicant: MiRus LLC, Marietta, GA (US)

(72) Inventors: Md Tausif Salim, Decatur, GA (US); Noah Roth, Marietta, GA (US)

(21) Appl. No.: 18/620,112

(22) Filed: Mar. 28, 2024

Related U.S. Application Data
(60) Provisional application No. 63/530,610, filed on Aug. 3, 2023.

(51) Int. Cl. A61F 2/24; B23P 11/00

(52) U.S. Cl. CPC A61F 2/2412; B23P 11/005

(57) ABSTRACT

Crimper systems, devices, and methods for reducing the crimped profile of a prosthetic heart valve by the non-simultaneous and/or non-uniform crimping of the valve frame during the crimping process. The crimper system is configured to facilitate in the desired folding profile of the leaflets of a prosthetic heart valve during the crimping of the frame of the prosthetic heart valve to reduce the number and volume of void spaces between and about the leaflets and to reduce the crimped outer diameter profile of the prosthetic heat valve, while reducing the risk of damage to the leaflets and other components of the prosthetic heart valve during the crimping process.

### REFERENCED APPLICATIONS

The present application claims priority to U.S. Provisional Application Ser. No. 63/530,610 filed Aug. 3, 2023, which is fully incorporated herein by reference.

### FIELD OF DISCLOSURE

The present disclosure relates generally to crimper systems, devices, and methods for reducing the crimped profile of a medical device, particularly to crimper systems, devices, and methods for reducing the crimped profile of a prosthetic heart valve, and even more particularly to crimper systems, devices, and methods that facilitate in the desired folding profile of the leaflets of a prosthetic heart valve during the crimping of the frame of the prosthetic heart valve to reduce the number and volume of void spaces between and about the leaflets and to reduce the crimped outer diameter profile of the prosthetic heat valve, while reducing the risk of damage to the leaflets and other components of the prosthetic heart valve during the crimping process. The crimper systems, devices, and methods for reducing the crimped profile of a prosthetic heart valve can be accomplished by the non-simultaneous and/or non-uniform crimping of the valve frame during the crimping process.

### BACKGROUND OF DISCLOSURE

Many cardiovascular devices such as expandable heart valves and the like are inserted into a patient via the vascular system of a patient and then expanded at the treatment site. These devices are typically crimped onto catheters prior to insertion into a patient. The minimum diameter to which the cardiovascular device can be crimped onto the catheter will set a limit to the size of the cardiovascular passageway (e.g., blood vessel) to which the cardiovascular device can be inserted. Smaller crimp diameters can result in reduced damage to a blood vessel and/or organ (e.g., heart, etc.) when inserting into or to and/or placing the cardiovascular device at the treatment site. Smaller crimp diameters can also allow the cardiovascular device to be placed in smaller diameter blood vessels (e.g., blood vessels located in the brain, etc.).

The crimp diameter of the expandable cardiovascular device can be reduced by reducing the thickness and/or size of the frame, struts, etc. of the cardiovascular device. However, such reduction in size also affects the strength of the cardiovascular device after being expanded. After the cardiovascular device is expanded, it must retain its expanded shape at the treatment area, otherwise the cardiovascular device could become dislodged from the treatment area, could damage the treatment area, and/or fail to properly function at the treatment area. As such, cardiovascular devices formed of tradition materials such as stainless steel (e.g., 316L: 17-19 wt. % chromium, 13-15 wt. % nickel, 2-4 wt. % molybdenum, 2 wt. % max manganese, 0.75 wt. % max silicon, 0.03 wt. % max carbon, balance iron) and cobalt-chromium alloys (e.g., MP35N: 19-21 wt. % chromium, 34-36 wt. % nickel, 9-11 wt. % molybdenum, 1 wt. % max iron, 1 wt. % max titanium, 0.15 wt. % max manganese, 0.15 wt. % max silicon, 0.025 wt. % max carbon, balance cobalt) are required to maintain a frame and/or strut size/thickness that limits how small of crimping diameter can be obtained by the crimped cardiovascular device. Other types of CoCr alloys that have been used are Phynox and Elgiloy alloy (38-42 wt. % cobalt, 18-22 wt. % chromium, 14-18 wt. % iron, 13-17 wt. % nickel, 6-8 wt. % molybdenum), and L605 alloy (18-22 wt. % chromium, 14-16 wt. % W, 9-11 wt. % nickel, balance cobalt), but have similar limitations as the other CoCr alloys.

Medical devices such as Transcatheter aortic valves (TAVs) represent a significant advancement in prosthetic heart valve technology. TAVs bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVs are disclosed in U.S. Pat. Nos. 5,411,522; 6,730,118; 10,729,543; 10,820,993; 10,856,970; 10,869,761; 10,952,852; 10,980,632; 10,980,633; and US Pub. No. 2020/0405482, all of which are incorporated fully herein by reference.

A TAV is designed to be compressed into a small diameter catheter, remotely placed within a patient's diseased aortic valve to take over the function of the native valve. Some TAVs are balloon-expandable, while others are self-expandable. In both cases, the TAVs are deployed within a calcified native valve that is forced permanently open and becomes the surface against which the frame is held in place by friction. TAVs can also be used to replace failing bioprosthetic or transcatheter valves, commonly known as a valve in valve procedure. Major TAVR advantages to the traditional surgical approaches include refraining from cardiopulmonary bypass, aortic cross-clamping and sternotomy which significantly reduces patients' morbidity.

However, several complications are associated with current TAV devices such as serious vascular injury or bleeding due to the large delivery profiles, mispositioning, crimp-induced leaflet damage, paravalvular leak, thrombosis, conduction system abnormalities and prosthesis-patient mismatch.

TAVR involves delivery, deployment, and implantation of a crimped, framed valve within a diseased aortic valve or degenerated bioprosthesis. Some limitation of the current procedure for TAVR include a) vascular complications such as dissection or severe bleeding due to the large size of the delivery system, b) high incidence of conduction system injury leading to permanent pacemaker implantation or sudden cardiac death; the conduction abnormalities are worsened by the frame recoil which necessitates that the operator reach a higher balloon inflation diameter to obtain a physiologic effective orifice area after balloon deflation, c) damage to the leaflets and/or frame during crimping of the frame of the prosthetic heart valve, and d) device failure. TAVR involves delivery, deployment, and implantation of a crimped valve frame within a diseased aortic valve or degenerated bioprosthesis. One limitation of these types of procedures is the diameter to which the valve frame can be crimped without damaging the leaflet tissues within, and vascular complications such as dissection due to the size of the delivery system.

As such, there has been an ongoing need for an improved medical device that can a) form smaller crimping diameters, b) obtain crimped diameter reductions while minimizing the damage to the leaflets during the crimping process, and/or c) addresses some of the deficiencies of prior art expandable devices such as, but not limited to, stents, TAVs and the like.

### SUMMARY OF THE DISCLOSURE

The present disclosure relates to crimper systems, devices, and methods for reducing the crimped profile of a medical such as, but not limited to, a prosthetic heart valve, and more particularly to crimper systems, devices, and methods that facilitate in the desired folding profile of the leaflets of a prosthetic heart valve during the crimping of the frame of the prosthetic heart valve to reduce the number and volume of void spaces between and about the leaflets and to reduce the crimped outer diameter profile of the prosthetic heat valve, while reducing the risk of damage to the leaflets and other components of the prosthetic heart valve during the crimping process. The crimper systems, devices, and methods for reducing the crimped profile of a prosthetic heart valve can be accomplished by the non-simultaneous and/or non-uniform crimping of the valve frame during the crimping process. Although the present disclosure will particularly discuss crimper systems, devices, and methods for use with prosthetic heart valves, it will be appreciated that the crimper systems, devices, and methods in accordance with the present disclosure can be used with other types of medical devices (e.g., stent, etc.) that include a frame that needs to be crimped, plastically deformed, etc. to a smaller profile.

In one non-limiting aspect of the present disclosure, there is provided a crimper device that is configured to reduce the outer diameter of the frame of the prosthetic heart valve along a longitudinal length of the prosthetic heart valve, wherein the crimper device reduces the frame either a) gradually (in a continuous manner) or b) stepwise (in a discreet manner) from i) the inflow side to the outflow side, or ii) from the outflow side to the inflow side, thereby causing a desired and/or uniform folding pattern of the leaflets in the prosthetic heart valve, and which desired and/or uniform leaflet folding pattern results in a reduced volume profile of the leaflets, which in turn results in a reduced outer diameter or profile of the crimped prosthetic heart valve. In either the continuous or stepwise crimping method, the inflow side portion or the outflow side portion can be partially or fully crimped prior to crimping the other portions of the frame of the prosthetic heart valve. In one non-limiting embodiment, the crimper device is used to reduce the outer diameter of the frame of the prosthetic heart valve along a longitudinal length of the prosthetic heart valve in a gradual or continuous manner from the inflow side to the outflow side of the prosthetic heart valve. In another non-limiting embodiment, the crimper device is used to reduce the outer diameter of the frame of the prosthetic heart valve along a longitudinal length of the prosthetic heart valve in stepwise manner from the inflow side to the outflow side of the prosthetic heart valve.

In another one non-limiting aspect of the present disclosure, there is provided a crimper device that includes a support assembly and a crimper assembly. In one on-limiting embodiment, the crimper assembly includes a first outer plate, a second outer plate, a first inner plate, a second inner plate, and a jaw assembly. The crimper device can include one or more handles to enable a user to manually operate the crimper device, or can include a control system that partially or fully automates the operation of the crimper device.

In another one non-limiting aspect of the present disclosure, there is provided a crimper device that is configured such that when the one or more handles are manually and/or automated operated, or the jaws of the crimper are caused to move by some other type of motorized operation, the first jaws and second jaws sequentially approach the center of the jaw assembly, wherein the center includes an opening wherein the prosthetic heart valve can be placed and crimped. As the first jaws and second jaws sequentially approach the center of the jaw assembly, the size or diameter or cross-sectional area of the opening reduces, thereby applying a crimping force on the prosthetic heart valve that is located in the opening. In one non-limiting configuration, the sequential movement of the first jaws and second jaws can optionally be configured to provide a linear relationship between the amount the handle is moved (e.g., distance handle is moved, etc.) and the diameter or cross-sectional area of the opening. In another non-limiting configuration, the sequential movement of the first jaws and second jaws can optionally be configured to provide a non-linear relationship between the amount the handle is moved (e.g., distance handle is moved, etc.) and the diameter or cross-sectional area of the opening. In one non-limiting configuration, the handle is configured to rotate about a handle axis. As the handle is rotated from the initial position (0° position) to the final position (e.g., 30-360° and all values and ranges therebetween), the movement of the handle about the handle axis causes a reduction in the diameter or cross-sectional area of the opening of the crimper device. In such an arrangement, for each degree of movement of the handle about the handle axis, the diameter or cross-sectional area of the opening of the crimper device can be caused proportionally reduce in size (e.g., a linear relationship between the handle rotational position and the size of the diameter or cross-sectional area of the opening) or reduce at some other rate (e.g., non-linear relationship between the handle rotational position and the size of the diameter or cross-sectional area of the opening). In another non-limiting configuration, the sequential movement of the first jaws and second jaws can optionally be configured to cause a first portion of the opening along the central longitudinal axis of the opening to reduce in diameter or cross-sectional area prior to a second portion of the opening along the central longitudinal axis of the opening to reduce in diameter or cross-sectional area. In another non-limiting configuration, the crimper device is configured to a) cause the inflow side portion of the prosthetic heart valve to be first partially or fully crimped, and thereafter the outflow side portion of the prosthetic heart valve to be partially or fully crimped, or b) cause the prosthetic heart valve to be progressively and continuously crimped along its longitudinal axis starting from the inflow end and thereafter progressing to and end at the outflow end of the prosthetic heart valve.

In another one non-limiting aspect of the present disclosure, there is provided a crimper device that is configured to a) progressively crimp a frame of the prosthetic heart valve from one end to the other end, b) progressively crimp a frame of the prosthetic heart valve from one portion to another portion of the frame, and c) batch crimping of different portions of the frame at different times during the crimping process (e.g., crimping a front portion of the frame prior to crimping the rear portion of the frame, etc.). Such crimping process has been found to facilitate in the folding of the leaflets in a prosthetic heart valve in an organized manner so that the frame can be crimped to smaller crimped profiles and damage to the leaflets during the crimping process is reduced.

In another one non-limiting aspect of the present disclosure, there is provided a crimper device wherein the manual movement of the handle of the crimper device can be replaced by a motorized movement mechanism. In such an arrangement, the motorized movement mechanism can be configured and/or controlled so as to a) move the handle at a generally constant speed from 1-100% (and all values and ranges therebetween) of the distance between the beginning position to the end position, b) move the handle at a generally non-constant speed from 1-100% (and all values and ranges therebetween) of the distance between the beginning position to the end position, or c) move the handle at some programmed speed profile as the handle moves between the beginning position to the end position.

In another one non-limiting aspect of the present disclosure, there is provided a crimper device that is configured to batch crimp the frame of the prosthetic heart valve at different portions of the frame at different times during the crimping process. In such an arrangement, the crimper device can a) include two handles wherein movement of the first handle causes only a portion of the jaw assembly of the crimper device to move to cause a first portion of the opening along the central longitudinal axis of the opening to reduce in diameter or cross-sectional area, and the second handle is configured to cause only a portion of the jaw assembly of the crimper device to move to cause a second portion of the opening along the central longitudinal axis of the opening to reduce in diameter or cross-sectional area, or b) includes a switching arrangement wherein when the single handle is initially moved, the causes only a portion of the jaw assembly of the crimper device to move to cause a first portion of the opening along the central longitudinal axis of the opening to reduce in diameter or cross-sectional area, and when the handle is moved back or near the beginning position, a switching arrangement is activated wherein when the handle is again moved the handle causes only a portion of the jaw assembly of the crimper device to move to cause a second portion of the opening along the central longitudinal axis of the opening to reduce in diameter or cross-sectional area or causes both portions of the opening along the central longitudinal axis of the opening to reduce in diameter or cross-sectional area. When a switching arrangement is optionally used, the switching arrangement can be a manual, a mechanical or an electro-mechanical switching arrangement.

In another one non-limiting aspect of the present disclosure, there is provided a leaflet folding device that can optionally be used with the crimper device in accordance with the present disclosure to facilitate in obtaining a leaflet folding configuration during the crimping of the frame of a prosthetic heart valve so as to obtain a desired folded leaflet configuration after the frame has been fully crimped. In one non-limiting embodiment, the leaflet folding device is configured to inwardly bend one or more or all of the end portions of the leaflets that are located at or near the outflow end of the prosthetic heart valve toward the central axis of the frame of the prosthetic heart valve. Such bending of the one or more leaflets by the leaflet folding device occurs a) prior to the initial crimping of the frame of the prosthetic heart valve, and/or b) during the crimping of the frame of the prosthetic heart valve. In another non-limiting method of use, prior to and/or during the crimping of the frame, a portion or all of the leaflet folding device is rotated (e.g., 2-180° and all values and ranges therebetween) about the longitudinal axis of the frame (e.g., central longitudinal axis of the frame, etc.) so as to facilitate in the folding of the leaflets during the crimping of the frame. Such rotation of the is used to facilitate in the folding of the leaflets. Such rotation of the one or more leaflets by the leaflet folding device occurs a) prior to the initial crimping of the frame of the prosthetic heart valve, and/or b) during the crimping of the frame of the prosthetic heart valve. One or more portions of the leaflet folding device can include a coating to a) limit or prevent damage to the frame and/or leaflets when one or more portions of the leaflet folding device engages the frame and/or leaflets during the crimping process, b) limit or prevent contamination of the frame and/or leaflets when one or more portions of the leaflet folding device engages the frame and/or leaflets during the crimping process, c) reduce friction between one or more portions of the leaflet folding device and the frame and/or leaflets during the crimping process, and/or d) form a surface color on one or more portions of the leaflet folding device to facilitate in visually positioning and using the leaflet folding device during the crimping process. The type and thickness of the coating, when used, is non-limiting.

In another one non-limiting aspect of the present disclosure, there is provided a leaflet folding device that can optionally be used with the crimper device in accordance with the present disclosure to facilitate in obtaining a leaflet folding configuration during the crimping of the frame of a prosthetic heart valve so as to obtain a desired folded leaflet configuration after the frame has been fully crimped and wherein the leaflet folding device is removed from or disengaged from the one or more leaflets prior to the outflow end of the frame of the prosthetic heart valve being 5-99% fully crimped (and all values and ranges therebetween). In another non-limiting method, the leaflet folding device is removed from or disengaged from the one or more leaflets prior to the outflow end of the frame of the prosthetic heart valve being 25-99% fully crimped. In another non-limiting method, the leaflet folding device is removed from or disengaged from the one or more leaflets prior to the outflow end of the frame of the prosthetic heart valve being 30-75% fully crimped. In another non-limiting method, the leaflet folding device is removed from or disengaged from the one or more leaflets after the outflow end of the frame of the prosthetic heart valve is fully crimped.

In accordance with another non-limiting aspect of the present disclosure, there is provided another device and method for folding the leaflets on a prosthetic heart valve that includes the step of inserting a radially collapsible insert inside the prosthetic heart valve prior to the partial crimping of the prosthetic heart valve. The prosthetic heart valve is then partially crimped (10-90% fully crimped and all values and ranges therebetween) while the radially collapsible insert is inside the prosthetic heart valve. Generally, the radially collapsible insert is inserted 50-100% (and all values and ranges therebetween) along the longitudinal length of the prosthetic heart valve. After the prosthetic heart valve is partially crimped, the radially collapsible insert is removed from the prosthetic heart valve and the prosthetic heart valve is then subjected to further crimping by the crimping device until the prosthetic heart valve is fully crimped. The radially collapsible insert can be designed to be reusable or be designed for a single use. The material used to form the radially collapsible insert is non-limiting (e.g., metal, plastic, paper, composite material, etc.). The cross-sectional shape of the radially collapsible insert prior to the insertion of the radially collapsible insert into the prosthetic heart valve can be constant or variable along the longitudinal length of the radially collapsible insert. During the crimping process involving the radially collapsible insert, the type of crimping process used to partially crimp the prosthetic heart valve while the radially collapsible insert is partially or fully inserted in the prosthetic heart valve is non-limiting. Such crimping processes can include a) a traditional prior art crimping process wherein the complete prosthetic heart valve is subjected to crimping forces, b) a stepwise crimping process wherein one portion of the prosthetic heart valve is subjected to crimping forces and then other portions of the prosthetic heart valve are subjected to crimping forces, and/or c) a progressive continuous crimping process wherein the crimping of the prosthetic heart valve starts at the inflow end or the outflow end and the crimping continuously progresses along the longitudinal length of the prosthetic heart valve to the opposite end of the prosthetic heart valve. After the radially collapsible insert is removed from the prosthetic heart valve, the crimping process used to complete the crimping of the prosthetic heart valve can include a) a traditional prior art crimping process wherein the complete prosthetic heart valve is subjected to crimping forces, b) a stepwise crimping process wherein one portion of the prosthetic heart valve is subjected to crimping forces and then other portions of the prosthetic heart valve are subjected to crimping forces, and/or c) a progressive continuous crimping process wherein the crimping of the prosthetic heart valve starts at the inflow end or the outflow end and the crimping continuously progresses along the longitudinal length of the prosthetic heart valve to the opposite end of the prosthetic heart valve. In one non-limiting method, during use of the radially collapsible insert while it is at least partially inserted in the prosthetic heart valve, the radially collapsible insert can be held or mounted to not rotate during the partial crimping of the prosthetic heart valve. In another non-limiting method, during use of the radially collapsible insert while it is at least partially inserted in the prosthetic heart valve, the radially collapsible insert can be rotated during the partial crimping of the prosthetic heart valve.

In accordance with another non-limiting aspect of the present disclosure, there is provided another device and method for folding the leaflets on a prosthetic heart valve that includes the step of inserting a radially collapsible insert inside the prosthetic heart valve prior to the partial crimping of the prosthetic heart valve wherein the cross-sectional shape of the radially collapsible insert prior to the insertion of the radially collapsible insert into the prosthetic heart valve is constant along the longitudinal length of the radially collapsible insert. The cross-sectional area of the radially collapsible insert prior to the insertion of the radially collapsible insert into the prosthetic heart valve can be constant or variable along the longitudinal length of the radially collapsible insert. In one non-limiting configuration, the cross-sectional area of the radially collapsible insert prior to the insertion of the radially collapsible insert into the prosthetic heart valve is constant along the longitudinal length of the radially collapsible insert.

In accordance with another non-limiting aspect of the present disclosure, there is provided another device and method for folding the leaflets on a prosthetic heart valve that includes the step of inserting a radially collapsible insert inside the prosthetic heart valve prior to the partial crimping of the prosthetic heart valve wherein the radially collapsible insert has a generally circular or oval cross-sectional shape along the longitudinal length of the radially collapsible insert. The radially collapsible insert can have a generally circular or oval cross-sectional shape along a portion (e.g., 10-99% and all values and ranges therebetween) or full longitudinal length of the radially collapsible insert. In one non-limiting configuration, the generally circular or oval cross-sectional shape of the radially collapsible insert has a generally constant cross-sectional size along 20-100% (and all values and ranges therebetween) the longitudinal length of the radially collapsible insert. In another non-limiting configuration, the radially collapsible insert has a hollow interior cavity along 20-100% (and all values and ranges therebetween) of the longitudinal length of the radially collapsible insert. In another non-limiting configuration, the hollow interior cavity has a generally circular or oval cross-sectional shape along a portion (e.g., 10-99% and all values and ranges therebetween) or full longitudinal length of the radially collapsible insert. The shape and/or cross-sectional area of the hollow interior cavity along the longitudinal length of the radially collapsible insert is generally constant; however, this is not required. The thickness of the walls and the material used to form the radially collapsible insert is selected such that the radially collapsible insert is caused to at least partially collapse when the prosthetic heart valve is partially crimped by a crimping device. Generally, the thickness of the wall of the radially collapsible insert is 0.1-10% (and all values and ranges therebetween) of the longest cross-sectional length or diameter of the radially collapsible insert. The radially collapsible insert is configured such that as the prosthetic heart valve is crimped, the leaflets will engage in the outer surface of the radially collapsible insert and be caused to initially fold in a certain way based on the engagement with the radially collapsible insert and the radial reduction of the frame of the prosthetic heart valve. During the partial crimping of the prosthetic heart valve, the radially collapsible insert is also caused to partially collapse due to the inwardly radial forces on the outer surface of the radially collapsible insert by the leaflets and the frame of the prosthetic heart valve. The partially collapsed shape of the radially collapsible insert also facilitates in the desired folding patterning of the leaflets as the prosthetic heart valve is crimped. Once the prosthetic heart valve is partially crimped, the radially collapsible insert is fully removed from the interior of the prosthetic heart valve and thereafter the prosthetic heart valve is subjected to a final crimping process to fully crimp the prosthetic heart valve. During the final crimping process, the leaflets are continued to be caused to fold in a desired manner to reduce or minimize the volume of the leaflets after the prosthetic heart valve is fully crimped. This continued desired leaflet folding is at least partially or fully the result of the initial folding of the leaflets when using the radially collapsible insert.

In accordance with another non-limiting aspect of the present disclosure, there is provided another device and method for folding the leaflets on a prosthetic heart valve that includes the step of inserting a radially collapsible insert inside the prosthetic heart valve prior to the partial crimping of the prosthetic heart valve wherein the radially collapsible insert has a plurality of arms extending outwardly from the outer surface of the radially collapsible insert. Generally, the number of arms on the radially collapsible insert is the same as the number of leaflets in the prosthetic heart valve; however, this is not required. The shape and/or cross-sectional area of the hollow interior cavity along the longitudinal length of the radially collapsible insert is generally constant; however, this is not required. The body and/or arms can optionally include a hollow interior cavity. When the body and/or arms include a hollow interior cavity, the hollow interior cavity extends 10-100% (and all values and ranges therebetween) of the longitudinal length of the radially collapsible insert. The thickness of the walls and the material used to form the radially collapsible insert is selected such that the radially collapsible insert is caused to at least partially collapse when the prosthetic heart valve is partially crimped by a crimping device. Generally, the thickness of the wall of the radially collapsible insert is 0.1-10% (and all values and ranges therebetween) of the longest cross-sectional length or diameter of the radially collapsible insert. Each of the arms can have the same or different size, shape, thickness, length, width, and/or configuration. Generally, two or more or all adjacently positioned arms can be equally spaced from one another about the outer circumference of the body of the radially collapsible insert; however, this is not required. Generally, the arms are formed of the same material as the body of the radially collapsible insert; however, this is not required. The length of each arm is generally 2-40 mm (and all values and ranges therebetween; 3-30 mm, etc.). Generally, one or more or all of the arms extend 60-100% (and all values and ranges therebetween) the longitudinal length of the radially collapsible insert. The angle α at which each arm initially extends from the outer surface of the body of the radially collapsible insert is 5-175° (and all values and ranges therebetween). The angle α for two or more or all arms can be the same or different. The shape of the arms extending from the outer surface of the body can be straight curved, wavy, etc. The number, shape, size, and configuration of the arms on the radially collapsible insert are selected to facilitate in the desired folding of the leaflets as the prosthetic heart valve is initially crimped. The radially collapsible insert with the one or more arms can be configured such that as the prosthetic heart valve is crimped, the leaflets will engage in the arms and the outer surface of the radially collapsible insert and be caused to initially fold in a certain way based on the engagement with the arms and outer surface of radially collapsible insert and the radial reduction of the frame of the prosthetic heart valve. During the partial crimping of the prosthetic heart valve, the radially collapsible insert is also caused to partially collapse due to the inwardly radial forces on the arm and/or outer surface of the radially collapsible insert by the leaflets and the frame of the prosthetic heart valve. The partially collapsed shape of the radially collapsible insert also facilitates in the desired folding patterning of the leaflets as the prosthetic heart valve is crimped. Once the prosthetic heart valve is partially crimped, the radially collapsible insert is fully removed from the interior of the prosthetic heart valve and thereafter the prosthetic heart valve is subjected to a final crimping process to fully crimp the prosthetic heart valve. During the final crimping process, the leaflets are continued to be caused to fold in a desired manner to reduce or minimize the volume of the leaflets after the prosthetic heart valve is fully crimped. This continued desired leaflet folding is at least partially or fully the result of the initial folding of the leaflets when using the radially collapsible insert. In one non-limiting configuration, the radially collapsible insert includes three arms. Each of the three arms has generally the same shape, thickness, length, width, and configuration. The three arms are spaced such that adjacently positioned arms are equally spaced from one another about the outer circumference of the body of the radially collapsible insert. Each of the arms extends 80-100% (and all values and ranges therebetween) the longitudinal length of the radially collapsible insert. Each of the arms has a slightly curve shape, a length of 3-10 mm (and all values and ranges therebetween), and an angle α that is 15-60° (and all values and ranges therebetween).

In accordance with another non-limiting aspect of the present disclosure, there is provided another device and method for folding the leaflets on a prosthetic heart valve that includes the step of inserting a forming shaft inside the prosthetic heart valve prior to the partial crimping of the prosthetic heart valve wherein the forming shaft has a plurality of arms extending outwardly from the outer surface of the forming shaft. Generally, the number of arms on the forming shaft is the same as the number of leaflets in the prosthetic heart valve; however, this is not required. One non-limiting embodiment, a portion of the forming shaft (e.g., the end portion, mid-portion, front portion, etc.) includes the plurality of arms. The plurality of arms can extend 1-100% (and all values and ranges therebetween) along the longitudinal length of the forming shaft. The forming shaft can have a body that is not limited in shape. Each of the arms can have the same or different size, shape, thickness, length, width, and/or configuration. Generally, two or more or all adjacently positioned arms are equally spaced from one another about the outer circumference of the body of the forming shaft; however, this is not required. Generally, the arms are formed of the same material as the body of the forming shaft; however, this is not required. The length of each arm is generally 2-40 mm (and all values and ranges therebetween; 3-30 mm, etc.). The angle at which each arm initially extends from the outer surface of the body of the radially collapsible insert is 5-175° (and all values and ranges therebetween). The angle for two or more or all arms can be the same or different. The shape of the arms that extend from the outer surface of the body can be straight curved, wavy, etc. The number, shape, size, and configuration of the arms on the forming shaft are selected to facilitate in the desired folding of the leaflets as the prosthetic heart valve is initially crimped. The forming shaft and/or one or more of the arms can be formed of a non-flexible or rigid material such that during the crimping of the prosthetic heart valve, the shaft and/or one or more of the arms flexes or bends 0-10% (and all values and ranges therebetween). In one non-limiting configuration, the shaft and/or one or more of the arms is formed of a hard plastic material, ceramic material, metal material, and/or composite material. The forming shaft with the one or more arms is configured such that as the prosthetic heart valve is crimped, the leaflets will engage in the arms and be caused to initially fold in a certain way based on the engagement with the arms and the radial reduction of the frame of the prosthetic heart valve. Once the prosthetic heart valve is partially crimped, the forming shaft is fully removed from the interior of the prosthetic heart valve and thereafter the prosthetic heart valve is subjected to a final crimping process to fully crimp the prosthetic heart valve. During the final crimping process, the leaflets are continued to be caused to fold in a desired manner to reduce or minimize the volume of the leaflets after the prosthetic heart valve is fully crimped. This continued desired leaflet folding is at least partially or fully the result of the initial folding of the leaflets when using the radially collapsible insert. In one non-limiting configuration, the forming shaft includes three arms. Each of the three arms has generally the same shape, thickness, length, width, and configuration. The three arms are spaced such that adjacently positioned arms are equally spaced from one another about the outer circumference of the body of the forming shaft. Each of the arms extends 5-50% (and all values and ranges therebetween) the longitudinal length of the forming shaft. Each of the arms has a slightly curve shape, a length of 3-10 mm (and all values and ranges therebetween), and an angle α that is 15-60° (and all values and ranges therebetween). After the forming shaft is removed from the prosthetic heart valve, the crimping processes that can be used to complete the crimping of the prosthetic heart valve can include a) a traditional prior art crimping process wherein the complete prosthetic heart valve is subjected to crimping forces, b) a stepwise crimping process wherein one portion of the prosthetic heart valve is subjected to crimping forces and then other portions of the prosthetic heart valve are subjected to crimping forces, and/or c) a progressive continuous crimping process wherein the crimping of the prosthetic heart valve starts at the inflow end or the outflow end and the crimping continuously progresses along the longitudinal length of the prosthetic heart valve to the opposite end of the prosthetic heart valve. In one non-limiting method, during use of the forming shaft while it is at least partially inserted in the prosthetic heart valve, the forming shaft can be held or mounted to not rotate during the partial crimping of the prosthetic heart valve. In another non-limiting method, during use of the forming shaft while it is at least partially inserted in the prosthetic heart valve, the forming shaft can be allowed to rotate during the partial crimping of the prosthetic heart valve.

In accordance with another non-limiting aspect of the present disclosure, there is provided another device and method for folding the leaflets on a prosthetic heart valve that includes the step of inserting a fold guide instrument at least partially in the interior of the prosthetic heart valve prior to the crimping of the prosthetic heart valve. The fold guide instrument includes two or more prongs that are configured to engage a portion of two or more leaflets as the prosthetic heart valve is partially crimped. The prongs of the fold guide instrument can be configured such that one or more or all of the prongs can be inserted 10-100% (and all values and ranges therebetween) along the longitudinal length of the prosthetic heart valve. The prongs can be configured that two or more or all of the prongs have the same shape, size, length, width, cross-sectional shape, and/or configuration. The prongs can be oriented that two or more of the adjacently positioned prongs have the same spacing from one another. In one non-limiting arrangement, the width and/or thickness of one or more or all of the prongs is 0.02-8 mm (and all values and ranges therebetween; 0.05-5 mm, etc.). The one or more prongs are generally configured to be flexible so the one or more prongs bend as the prosthetic heart valve is partially crimped. The material used to form the fold guide instrument is non-limiting (e.g., metal, plastic, paper, composite material, etc.). During use, the prongs of the fold guide instrument are inserted into the interior of the prosthetic heart valve along a portion or the complete longitudinal length of the prosthetic heart valve prior to crimping the prosthetic heart valve. As the prosthetic heart valve is partially crimped, the prongs on the fold guide instrument engage one or more leaflets and cause the leaflets to initially fold in a certain way based on the engagement of the leaflets with the prongs of the fold guide instrument. During the partial crimping of the prosthetic heart valve, the prongs of the fold guide instrument are caused to partially bend due to the inwardly radial forces on the prongs by the leaflets and/or the frame of the prosthetic heart valve. Once the prosthetic heart valve is partially crimped, the fold guide instrument is removed from the interior of the prosthetic heart valve and thereafter the prosthetic heart valve is subjected to a final crimping process to fully crimp the prosthetic heart valve. During the final crimping process, the leaflets are continued to be caused to fold in a desired manner to reduce or minimize the volume of the leaflets after the prosthetic heart valve is fully crimped. This continued desired leaflet folding is at least partially or fully the result of the initial folding of the leaflets when using the fold guide instrument. During the crimping process that involves the use of the fold guide instrument, the type of crimping process used to partially crimp the prosthetic heart valve while the radially collapsible insert is partially or fully inserted in the prosthetic heart valve is non-limiting. Such crimping processes can include a) a traditional prior art crimping process wherein the complete prosthetic heart valve is subjected to crimping forces, b) a stepwise crimping process wherein one portion of the prosthetic heart valve is subjected to crimping forces and then other portions of the prosthetic heart valve are subjected to crimping forces, and/or c) a progressive continuous crimping process wherein the crimping of the prosthetic heart valve starts at the inflow end or the outflow end and the crimping continuously progresses along the longitudinal length of the prosthetic heart valve to the opposite end of the prosthetic heart valve. After the fold guide instrument is removed from the prosthetic heart valve, the crimping processes that can be used to complete the crimping of the prosthetic heart valve can include a) a traditional prior art crimping process wherein the complete prosthetic heart valve is subjected to crimping forces, b) a stepwise crimping process wherein one portion of the prosthetic heart valve is subjected to crimping forces and then other portions of the prosthetic heart valve are subjected to crimping forces, and/or c) a progressive continuous crimping process wherein the crimping of the prosthetic heart valve starts at the inflow end or the outflow end and the crimping continuously progresses along the longitudinal length of the prosthetic heart valve to the opposite end of the prosthetic heart valve. In one non-limiting method, during use of the fold guide instrument while it is at least partially inserted in the prosthetic heart valve, the fold guide instrument can be held or mounted to not rotate during the partial crimping of the prosthetic heart valve. In another non-limiting method, during use of the fold guide instrument while it is at least partially inserted in the prosthetic heart valve, the fold guide instrument can be allowed to rotate during the partial crimping of the prosthetic heart valve. In non-limiting embodiment, the fold guide instrument includes three prongs. Each of the three prongs has generally the same shape, thickness, length, width, and configuration. The three prongs are spaced such that adjacently positioned prongs are equally spaced from one another. Each of the prongs is configured to extend 10-100% (and all values and ranges therebetween) the longitudinal length of the interior of the prosthetic heart valve. Each of the prongs has a thickness and/or width of 0.05-3 mm (and all values and ranges therebetween).

In another one non-limiting aspect of the present disclosure, there is provided a leaflet folding device that can optionally be used with the crimper device in accordance with the present disclosure to facilitate in obtaining a leaflet folding configuration during the crimping of the frame of a prosthetic heart valve, and wherein the leaflet folding device includes a handle portion and one or more leaflet engagement members that are attached to and extend from the handle portion. In one non-limiting configuration, the handle portion is sized and shaped such that it can be grasped by a user to enable the user to position the one or more leaflet engagement members into a portion of the prosthetic heart valve to cause the bending of the one or more leaflets. The shape and length and material and size of the handle portion are non-limiting. In one non-limiting specific configuration, the handle portion has a cylindrical shape and a length of 1-12 inches (and all values and ranges therebetween) and a cross-sectional area that is 10-95% (and all values and ranges therebetween) of the cross-sectional area of the outflow end of the frame prior to the crimping of the frame (e.g., the cross-sectional area of the outflow end of the frame in the fully expanded position, etc.). In another non-limiting configuration, the one or more leaflet engagement members that are attached to the distal end or distal end portion of the handle portion. The type of connection used to attach the one or more leaflet engagement members to the handle is non-limiting. In one non-limiting configuration, the one or more leaflet engagement members extend radially outwardly (e.g., 5-45° radially outwardly and all values and ranges therebetween) from the central longitudinal axis of the handle portion. When the leaflet folding device includes two or more leaflet engagement members, two or more or all of the leaflet engagement members can have the same size, shape, and/or be formed of the same material; however, this is not required. In another non-limiting configuration, one or more of the leaflet engagement members are formed of a flexible material that enables the one or more leaflet engagement members to flex and/or bend a) when positioning the one or more leaflet engagement members about one or more leaflets, and/or b) during the crimping of the frame of the prosthetic heart valve and while the one or more leaflet engagement members are still engaged with the one or more leaflets during the crimping of the frame. In another non-limiting configuration, the one or more leaflet engagement members are each formed of a wire loop (e.g., metal wire loop, plastic wire loop, etc.). In another non-limiting embodiment, the leaflet folding device is moved along the longitudinal axis of the frame and toward the frame until the one or more leaflet engagement members engage the end or end portion of the one or more leaflets. Thereafter, the leaflet folding device continued to be moved along the longitudinal axis of the frame such that the end or end portion of the one or more leaflet engagement members move between the one or more leaflets and the inner surface of the frame. As the leaflet folding device is continued to be moved along the longitudinal axis of the frame, the angular orientation of the one or more leaflet engagement members relative to the central axis of the handle portion of the leaflet folding device causes the end and end portions of the leaflets to be bent toward the central axis of the frame. Generally, the size and configuration of the end region of the one or more leaflet engagement members inhibits or prevents the end or end region of the one or more leaflet engagement members from passing through the side openings in the frame of the prosthetic heart valve. Furthermore, the one or more leaflet engagement members generally are inserted only through a portion of the longitudinal length of the frame (e.g., 1-80% of the longitudinal length and all values and ranges therebetween). In one non-limiting method of use, one or more leaflet engagement members are inserted only through a portion of the longitudinal length of the frame and are spaced form the region of the frame wherein the one or more leaflets are connected to the frame.

In another non-limiting aspect of the present disclosure, there is provided a leaflet folding device that can optionally be used with the crimper device in accordance with the present disclosure to facilitate in obtaining a leaflet folding configuration during the crimping of the frame of a prosthetic heart valve so as to obtain a desired folded leaflet configuration after the frame has been fully crimped, and wherein the leaflet folding device includes a cylindrical body portion that is configured to be partially or fully inserted into the prosthetic device a) prior to the initial crimping of the frame of the prosthetic heart valve, and/or b) during the crimping of the frame of the prosthetic heart valve. The cylindrical body portion can be a) a solid structure, b) a hollow structure that includes a cavity that extends partially or fully along the longitudinal length of the cylindrical body, or c) a cylindrical body that includes one or more internal void spaces and/or surface cavities. The diameter and/or cross-sectional area of the portion of the cylindrical body portion that is configured to be inserted into the prosthetic heart valve and can a) be constant along 50-100% (and all values and ranges therebetween) of the longitudinal length of such cylindrical body portion, or b) vary along the longitudinal length of such cylindrical body portion. The cylindrical body portion can optionally be formed of a flexible material (e.g., plastic material, foam material, etc.). In one non-limiting configuration, the outer diameter of the cylindrical body portion of the leaflet folding device that is configured to be partially or fully inserted into the prosthetic device is about 5-90% (and all values and ranges therebetween) of the inner diameter of the frame of the prosthetic heart valve prior to being crimped. In another non-limiting method, the cylindrical body portion of the leaflet folding device is removed from the frame of the prosthetic heart valve prior to the inner diameter of the frame of the prosthetic heart being reduced during the crimping process to a diameter that is 0-20% (and all values and ranges therebetween) greater than the outer diameter of the cylindrical body portion of the leaflet folding device. In one non-limiting method of use, the cylindrical body portion of the leaflet folding device is positioned within the region of the leaflets such that the leaflets are located between the cylindrical body portion of the leaflet folding device and the frame. During the crimping of the frame of the prosthetic heart valve, the cylindrical body portion of the leaflet folding device can be a) moved along the longitudinal axis of the frame, and/or b) rotated about the longitudinal axis of the frame.

One non-limiting object of the present disclosure is the provision of a device and method for crimping a crimpable or plastically deformable portion of a medical device.

Another and/or alternative non-limiting object of the present disclosure is the provision of a device and method for crimping a crimpable or plastically deformable portion of a medical device that includes one or more leaflets.

Another and/or alternative non-limiting object of the present disclosure is the provision of a device and method for crimping a crimpable or plastically deformable portion of a medical device that includes one or more leaflets so as to reduce void spaces about the leaflets after completion of the crimping of the crimpable or plastically deformable portion of a medical device.

Another and/or alternative non-limiting object of the present disclosure is the provision of a device and method for crimping a crimpable or plastically deformable portion of a medical device that includes one or more leaflets so as to obtains a desired folding profile of the leaflets after completion of the crimping of the crimpable or plastically deformable portion of a medical device.

Another and/or alternative non-limiting object of the present disclosure is the provision of a device and method for crimping a crimpable or plastically deformable portion of a medical device that includes one or more leaflets so as to facilitate in the desired folding profile of the leaflets of a prosthetic heart valve during the crimping of the frame of the prosthetic heart valve to reduce the number and volume of void spaces between and about the leaflets and to reduce the crimped outer diameter profile of the prosthetic heat valve, while reducing the risk of damage to the leaflets and other components of the prosthetic heart valve during the crimping process.

Another and/or alternative non-limiting object of the present disclosure is the provision of a device and method for crimping a crimpable or plastically deformable portion of a medical device that includes one or more leaflets so as to reduce the outer diameter or cross-sectional area of the portion of the crimpable or plastically deformable portion of a medical device that includes the leaflets.

Another and/or alternative non-limiting object of the present disclosure is the provision of a method for reducing the profile of a frame of a medical device comprising a) providing a medical device that includes a crimpable or plastically deformable portion; b) providing a crimping device; the crimping device includes a crimping assembly having a device opening; the device opening is configured to receive at least a portion of the medical device; the device opening is configured to reduce in diameter or cross-sectional area during operation of the crimping device; c) inserting at least a portion of the crimpable or plastically deformable portion of the medical device into the device opening; and d) operating the medical device to cause at least a portion of the device opening to reduce in diameter or cross-sectional area to thereby exert a crimping force initially on only a first portion of the crimpable or plastically deformable portion of the medical device which causes the first portion of the crimpable or plastically deformable portion of the medical device to reduce in cross-sectional area, and thereafter continuing to use the crimping device to subsequently exert a crimping force on a second portion of the crimpable or plastically deformable portion of the medical device which causes the second portion of the crimpable or plastically deformable portion of the medical device to reduce in cross-sectional area.

Another and/or alternative non-limiting object of the present disclosure is the provision of a method for reducing the profile of a frame of a medical device wherein the crimping device is configured to continue to exert the crimping force on both the first and second portions of the crimpable or plastically deformable portion of the medical device after initially exerting the crimping force on the second portion so as to further reduce the diameter or cross-sectional area of both the first and second portions of the crimpable or plastically deformable portion of the medical device.

Another and/or alternative non-limiting object of the present disclosure is the provision of a method for reducing the profile of a frame of a medical device wherein the crimping device is configured to exert the crimping force on the first portion until the diameter or cross-sectional area of the first portion of the crimpable or plastically deformable portion of the medical device is reduced by at least 1% prior to the crimping device initially exerting the crimping force on the second portion of the crimpable or plastically deformable portion of the medical device.

Another and/or alternative non-limiting object of the present disclosure is the provision of a method for reducing the profile of a frame of a medical device wherein the crimping device is configured to exert the crimping force on the first portion until the diameter or cross-sectional area of the first portion of the crimpable or plastically deformable portion of the medical device is reduced by at least 5% prior to the crimping device initially exerting the crimping force on the second portion of the crimpable or plastically deformable portion of the medical device.

Another and/or alternative non-limiting object of the present disclosure is the provision of a method for reducing the profile of a frame of a medical device wherein the crimping device is configured to exert the crimping force on the first portion until the diameter or cross-sectional area of the first portion of the crimpable or plastically deformable portion of the medical device is reduced by at least 25% prior to the crimping device initially exerting the crimping force on the second portion of the crimpable or plastically deformable portion of the medical device.

Another and/or alternative non-limiting object of the present disclosure is the provision of a method for reducing the profile of a frame of a medical device wherein the crimping device is configured to exert the crimping force on the first portion until the diameter or cross-sectional area of the first portion of the crimpable or plastically deformable portion of the medical device is reduced by at least 50% prior to the crimping device initially exerting the crimping force on the second portion of the crimpable or plastically deformable portion of the medical device.

Another and/or alternative non-limiting object of the present disclosure is the provision of a method for reducing the profile of a frame of a medical device wherein the first and second portion of the crimpable or plastically deformable portion of the medical device are both simultaneously positioned in the device opening during reduction of the diameter or cross-sectional area of the first and second portions of the crimpable or plastically deformable portion of the medical device by the crimper device.

Another and/or alternative non-limiting object of the present disclosure is the provision of a method for reducing the profile of a frame of a medical device wherein the first portion of the crimpable or plastically deformable portion of the medical device includes an inflow end portion of the medical device.

Another and/or alternative non-limiting object of the present disclosure is the provision of a method for reducing the profile of a frame of a medical device wherein the second portion of the crimpable or plastically deformable portion of the medical device includes an outflow end portion of the medical device.

Another and/or alternative non-limiting object of the present disclosure is the provision of a method for reducing the profile of a frame of a medical device wherein the crimping assembly includes a first set of jaws and a second set of jaws; the first set of jaws includes first and second jaws; the second set of jaws includes first and second jaws; the first jaw of the first and second set of jaws are positioned adjacent to one another and slidably connected to one another to enable the first jaw of the first and second set of jaws to independently move with respect to one another while being connected to one another; the second jaw of the first and second set of jaws are positioned adjacent to one another and slidably connected to one another to enable the second jaw of the first and second set of jaws to independently move with respect to one another while being connected to one another.

Another and/or alternative non-limiting object of the present disclosure is the provision of a method for reducing the profile of a frame of a medical device wherein the crimper device includes a support assembly that supports the crimper assembly; the crimper assembly further includes a first outer plate, a second outer plate, a first inner plate, and a second inner plate; the first inner plate engages each of the jaws of the first set of jaws of the crimping assembly; each of the jaws of the first set of jaws are independently movable relative to the first inner plate; the second inner plate engages each of the jaws of the second set of jaws of the crimping assembly; each of the jaws of the second set of jaws are independently movable relative to the second inner plate; the first inner plate is positioned on one side of the crimping assembly and the second inner plate is positioned on an opposite side of the crimping assembly.

Another and/or alternative non-limiting object of the present disclosure is the provision of a method for reducing the profile of a frame of a medical device wherein the first and second inner plates are non-rotatably connected to the support assembly; the first and second outer plates are rotatably connected to the support assembly; the first outer plate engages each of the jaws of the first set of jaws of the crimping assembly; the second outer plate engages each of the jaws of the second set of jaws of the crimping assembly.

Another and/or alternative non-limiting object of the present disclosure is the provision of a method for reducing the profile of a frame of a medical device wherein the first and second jaws of the first set of jaws includes a pin or flange that at least partially forms an engagement with both the first inner plate and the first outer plate; the first and second jaws of the second set of jaws includes a pin or flange that at least partially forms an engagement with both the second inner plate and the second outer plate.

Another and/or alternative non-limiting object of the present disclosure is the provision of a method for reducing the profile of a frame of a medical device wherein each of the first and second inner plates include a plurality of slots; each of the slots of the first inner plate is configured to receive the pin or flange from one of the jaws of the first set of jaws; each of the pin or flange of each of the jaws of the first set of jaws is configured to move within the respective slot when the first outer plate is moved relative to the support assembly; movement of the pin or flange of each of the jaws of the first set of jaws within the respective slot of the first inner plate results in the first set of jaws causing a portion of the device opening to reduce in diameter or cross-sectional area; each of the pin or flange of each of the jaws of the second set of jaws is configured to move within the respective slot when the second outer plate is moved relative to the support assembly; movement of the pin or flange of each of the jaws of the second set of jaws within the respective slot of the second inner plate results in the second set of jaws causing a portion of the device opening to reduce in diameter or cross-sectional area.

Another and/or alternative non-limiting object of the present disclosure is the provision of a method for reducing the profile of a frame of a medical device wherein the medical device includes one or more leaflets that are connected to the crimpable or plastically deformable portion of the medical device.

Another and/or alternative non-limiting object of the present disclosure is the provision of a method for reducing the profile of a frame of a medical device wherein the medical device is a prosthetic heart valve; the crimpable or plastically deformable portion of the medical device includes a frame of the prosthetic heart valve; the one or more leaflets are connected to the frame.

Another and/or alternative non-limiting object of the present disclosure is the provision of a method for reducing the profile of a frame of a medical device further including the step of causing at least a portion of one or more of the leaflets to be bent toward a central longitudinal axis of the crimpable or plastically deformable portion of the medical device a) prior to applying the crimping force on the crimpable or plastically deformable portion of the medical device, and/or b) while applying the crimping force to the crimping of the crimpable or plastically deformable portion of the medical device.

Another and/or alternative non-limiting object of the present disclosure is the provision of a method for reducing the profile of a frame of a medical device further includes the step of applying a rotational force about a central longitudinal axis of the crimpable or plastically deformable portion to one or more of the leaflets a) prior to applying the crimping force on the crimpable or plastically deformable portion of the medical device, and/or b) while applying the crimping force to the crimping of the crimpable or plastically deformable portion of the medical device.

Another and/or alternative non-limiting object of the present disclosure is the provision of a method for reducing the profile of a frame of a medical device further providing a leaflet folding device that is configured to facilitate in folding the one or more of the leaflets during the step of applying the crimping force on the crimpable or plastically deformable portion of the medical device.

Another and/or alternative non-limiting object of the present disclosure is the provision of a method for reducing the profile of a frame of a medical device further providing a leaflet folding device that is configured to facilitate in folding the one or more of the leaflets during the step of applying the crimping force on the crimpable or plastically deformable portion of the medical device, wherein the leaflet folding device is positioned within the leaflets such that the leaflets are positioned between the frame of the prosthetic heat valve and the leaflet folding device.

Another and/or alternative non-limiting object of the present disclosure is the provision of a method for reducing the profile of a frame of a medical device further providing a leaflet folding device that is configured to facilitate in folding the one or more of the leaflets during the step of applying the crimping force on the crimpable or plastically deformable portion of the medical device, wherein the leaflet folding device is positioned within the leaflets such that the leaflets are positioned between the frame of the prosthetic heat valve and the leaflet folding device, and wherein the leaflet folding device includes a) a hollow, collapsible body portion that is configured to be inserted into the prosthetic heart valve during the crimping process, b) one or more arms that radially extend from the body portion of the leaflet folding device, c) a hollow, collapsible body portion that is configured to be inserted into the prosthetic heart valve during the crimping process, and which includes one or more arms that are collapsible and that radially extend from the body portion of the leaflet folding device, d) a hollow, collapsible body portion that is configured to be inserted into the prosthetic heart valve during the crimping process, and which includes one or more arms that are collapsible and that radially extend from the body portion of the leaflet folding device, e) a non-collapsible body portion and one or more non-collapsible arms that radially extend from the body portion of the leaflet folding device, or f) a body portion that includes two or more prongs that are configured to engage a portion of two or more leaflets.

Another and/or alternative non-limiting object of the present disclosure is the provision of a method for reducing the profile of a frame of a medical device further including the step of positioning at least a portion of the leaflet folding device between the one or more leaflets and the crimpable or plastically deformable portion of the medical device to facilitate in a) bending at least a portion of one or more of the leaflets toward the central longitudinal axis of the crimpable or plastically deformable portion, and/or b) rotating at least a portion of one or more of the leaflets about the central longitudinal axis of the crimpable or plastically deformable portion.

Another and/or alternative non-limiting object of the present disclosure is the provision of a method for reducing the profile of a frame of a medical device further includes the step of fully disengaging the leaflet folding device from one or more of the leaflets prior to completion of applying the crimping force on the crimpable or plastically deformable portion of the medical device.

Another and/or alternative non-limiting object of the present disclosure is the provision of a method for reducing the profile of a frame of a medical device wherein the leaflet folding device includes a handle portion and one or more leaflet engagement members that are attached to and extend from the handle portion; the one or more leaflet engagement members are at least partially formed of a flexible material that enables the one or more leaflet engagement members to flex and/or bend a) when positioning the one or more leaflet engagement members about one or more leaflets, and/or b) as the crimpable or plastically deformable portion of the medical device reduces in diameter or cross-sectional area as the crimping force is applied on the crimpable or plastically deformable portion of the medical device.

Another and/or alternative non-limiting object of the present disclosure is the provision of a method for reducing the profile of a frame of a medical device wherein the one or more leaflet engagement members include a wire loop.

Another and/or alternative non-limiting object of the present disclosure is the provision of a method for reducing the profile of a frame of a medical device further including the step of positioning a portion or one or more of the leaflet engagement members between a portion of one or more of the leaflets and the crimpable or plastically deformable portion of the medical device to cause at least a portion of the one or more of the leaflets to bend towards the central longitudinal axis of the crimpable or plastically deformable portion of the medical device.

Another and/or alternative non-limiting object of the present disclosure is the provision of a crimper device that is configured to crimp a crimpable or plastically deformable portion of a medical device; the crimping device includes a crimping assembly having a device opening; the device opening is configured to receive at least a portion of the medical device; the device opening is configured to reduce in diameter or cross-sectional area during operation of the crimping device; a first portion of the device opening is configured to a) reduce in diameter or cross-sectional area at a different rate as compared to a second portion of the device opening during the operation of the crimping device, and/or b) begin reducing in diameter or cross-sectional area at a different time as compared to a second portion of the device opening during the operation of the crimping device.

Another and/or alternative non-limiting object of the present disclosure is the provision of a crimper device that is configured to crimp a crimpable or plastically deformable portion of a medical device wherein the crimping assembly includes a first set of jaws and a second set of jaws; the first set of jaws includes first and second jaws; the second set of jaws includes first and second jaws; the first jaw of the first and second set of jaws are positioned adjacent to one another and slidably connected to one another to enable the first jaw of the first and second set of jaws to independently move with respect to one another while being connected to one another; the second jaw of the first and second set of jaws are positioned adjacent to one another and slidably connected to one another to enable the second jaw of the first and second set of jaws to independently move with respect to one another while being connected to one another.

Another and/or alternative non-limiting object of the present disclosure is the provision of a crimper device that is configured to crimp a crimpable or plastically deformable portion of a medical device further including a support assembly that supports the crimper assembly; the crimper assembly further includes a first outer plate, a second outer plate, a first inner plate, and a second inner plate; the first inner plate engages each of the jaws of the first set of jaws of the crimping assembly; each of the jaws of the first set of jaws are independently movable relative to the first inner plate; the second inner plate engages each of the jaws of the second set of jaws of the crimping assembly; each of the jaws of the second set of jaws are independently movable relative to the second inner plate; the first inner plate is positioned on one side of the crimping assembly and the second inner plate is positioned on an opposite side of the crimping assembly.

Another and/or alternative non-limiting object of the present disclosure is the provision of a crimper device that is configured to crimp a crimpable or plastically deformable portion of a medical device wherein the first and second inner plates are non-rotatably connected to the support assembly; the first and second outer plates are rotatably connected to the support assembly; the first outer plate engages each of the jaws of the first set of jaws of the crimping assembly; the second outer plate engages each of the jaws of the second set of jaws of the crimping assembly.

Another and/or alternative non-limiting object of the present disclosure is the provision of a crimper device that is configured to crimp a crimpable or plastically deformable portion of a medical device wherein the first and second jaws of the first set of jaws includes a pin or flange that at least partially forms an engagement with both the first inner plate and the first outer plate; the first and second jaws of the second set of jaws includes a pin or flange that at least partially forms an engagement with both the second inner plate and the second outer plate.

Another and/or alternative non-limiting object of the present disclosure is the provision of a crimper device that is configured to crimp a crimpable or plastically deformable portion of a medical device wherein each of the first and second inner plates include a plurality of slots; each of the slot of the first inner plate is configured to receive the pin or flange from one of the jaws of the first set of jaws; each of the pin or flange of each of the jaws of the first set of jaws is configured to move within the respective slot when the first outer plate is moved relative to the support assembly; movement of the pin or flange of each of the jaws of the first set of jaws within the respective slot of the first inner plate results in the first set of jaws causing a portion of the device opening to reduce in diameter or cross-sectional area; each of the pin or flange of each of the jaws of the second set of jaws is configured to move within the respective slot when the second outer plate is moved relative to the support assembly; movement of the pin or flange of each of the jaws of the second set of jaws within the respective slot of the second inner plate results in the second set of jaws causing a portion of the device opening to reduce in diameter or cross-sectional area.

Another and/or alternative non-limiting object of the present disclosure is the provision of a method for reducing the outer diameter of a crimped prosthetic heart valve comprising a) providing a prosthetic heart valve; the prosthetic heart valve includes a frame, a leaflet structure supported by the frame; the frame in a non-fully crimped state; b) providing a diameter reducing device that is configured to reduce an outer diameter of the prosthetic heart valve; c) at least partially inserting the prosthetic heart valve in the diameter reducing device; and d) initially reducing an outer diameter of the prosthetic heart valve by use of the diameter reducing device; and wherein the step of reducing includes applying a crimping force on the frame of the prosthetic heart valve along the longitudinal length of the prosthetic heart valve; and wherein the step of reducing includes A) gradual application of the crimping force in a continuously progressive manner along a longitudinal length of the frame by i) initially applying the crimping force at an inflow end of the frame and subsequently applying the crimping force at locations that are spaced from the inflow end of the frame until the crimping force is applied to a complete outer surface of the frame; or ii) initially applying the crimping force at an outflow end of the frame and subsequently applying the crimping force at locations that are spaced from the outflow end of the flame until the crimping force is applied to a complete outer surface of the frame; or B) stepwise application of the crimping force along a longitudinal length of the frame by i) initially applying the crimping force at an inflow region of the frame until a portion or all of the inflow region of the frame is crimped to 10-100% (and all values and ranges therebetween) of a fully crimped diameter or cross-sectional area, and then subsequently applying the crimping force at one or more locations that are spaced from the inflow region of the frame to crimped the one or more locations that are spaced from the inflow region of the frame to 10-100% (and all values and ranges therebetween) of a fully crimped diameter or cross-sectional area, and wherein the inflow region of the frame extends from an inflow end of the frame to 0.1-75% (and all values and ranges therebetween) of a longitudinal length of the frame; or ii) initially applying the crimping force at an outflow side of the frame until a portion or all of a outflow region of the frame is crimped to 10-100% (and all values and ranges therebetween) of a fully crimped diameter or cross-sectional area, and then subsequently applying the crimping force at one or more locations that are spaced from the outflow region of the frame to crimped the one or more locations that are spaced from the outflow region of the frame to 10-100% (and all values and ranges therebetween) of a fully crimped diameter or cross-sectional area, and wherein the outflow region of the frame extends from an outflow end of the frame to 0.1-75% (and all values and ranges therebetween) of a longitudinal length of the frame.

Another and/or alternative non-limiting object of the present disclosure is the provision of a method for reducing the profile of a frame of a medical device; the method comprising a) providing a medical device that includes a crimpable or plastically deformable portion; b) providing a crimping device; the crimping device includes a crimping assembly having a device opening; the device opening is configured to receive at least a portion of the medical device; the device opening is configured to reduce in diameter or cross-sectional area during operation of the crimping device; c) inserting at least a portion of the crimpable or plastically deformable portion of the medical device into the device opening; and d) operating the medical device to cause at least a portion of the device opening to reduce in diameter or cross-sectional area to thereby exert a crimping force initially on only a first portion of the crimpable or plastically deformable portion of the medical device which causes the first portion of the crimpable or plastically deformable portion of the medical device to reduce in cross-sectional area, and thereafter continuing to use the crimping device to subsequently exert a crimping force on a second portion of the crimpable or plastically deformable portion of the medical device which causes the second portion of the crimpable or plastically deformable portion of the medical device to reduce in cross-sectional area; the first portion of the crimpable or plastically deformable portion of the medical device constitutes 0.01%-75% of a longitudinal length of the crimpable or plastically deformable portion of the medical device.

These and other objects and advantages will become apparent to those skilled in the art upon reading and following the description taken together with the accompanying drawings.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 depicts a non-limiting implementation of the disclosed crimper device shown in an exploded configuration, wherein the crimper device includes a support assembly and a crimper assembly having a first outer plate, a second outer plate, a first inner plate, a second inner plate, and a jaw assembly;

FIG. 2 is a perspective view of the crimper device of FIG. 1, shown in a partially exploded configuration;

FIG. 3A is a perspective view of the crimper device of FIG. 1, shown in a fully assembled configuration;

FIG. 3B is a side view of the crimper device of FIG. 1, shown in a fully assembled configuration;

FIGS. 4A-4B illustrate the support assembly of FIG. 1, wherein FIG. 4A is a perspective view of the support assembly, and wherein FIG. 4B is a top view of the support assembly;

FIGS. 5A-5B illustrate the first outer plate of FIG. 1, wherein FIG. 5A is an external view of the first outer plate, and wherein FIG. 5B is an internal view of the first outer plate;

FIG. 6 illustrates an internal view of the second outer plate of FIG. 1;

FIG. 7 is a perspective view of the jaw assembly of FIG. 1, wherein the jaw assembly comprises a first jaw and a second jaw;

FIG. 8 illustrates the first jaw of the jaw assembly of FIG. 7;

FIG. 9 illustrates the second jaw of the jaw assembly of FIG. 7;

FIG. 10 is a perspective view of the first jaw of FIG. 8 partially movably coupled to the second jaw of FIG. 9;

FIG. 11 is a perspective view of the first jaw of FIG. 8 fully movably coupled to the second jaw of FIG. 9;

FIG. 12 is a perspective view of an exemplary pin for use with the jaw assembly of FIG. 7;

FIGS. 13A-13C are perspective views of the crimper device of FIG. 1 depicting the exemplary movement of the jaw assembly from a partially opened position to a partially closed position;

FIGS. 14A-14B are perspective views of the crimper device of FIG. 1 depicting the exemplary movement of the jaw assembly from a partially closed position to a partially opened position;

FIG. 15 is a line graph showing the linear relationship between the rotational angle of the handle on the crimper device and the diameter of an opening of the jaw assembly formed by the first and second jaw as the handle is moved from the handle beginning position (0°) to the end position (160°);

FIG. 16 illustrates the folding profile of leaflets in a partially crimped prosthetic heart valve by use of the crimping process in accordance with the present disclosure wherein the leaflets are being to align so that void spaces are reduced once the frame is fully crimped;

FIG. 17 illustrates the folding profile of leaflets in the fully crimped prosthetic heart valve, wherein the prosthetic heart valve was crimped by the crimper device in accordance with the present disclosure and wherein the leaflets were caused to be folded in accordance with the one or more methods of the present disclosure, and wherein there is a reduction of void spaces about the folded leaflets when the frame valve is in the fully crimped state;

FIGS. 18A-C illustrates one non-limiting crimping method for progressively or stepwise crimping a prosthetic heart valve to obtain a leaflet folding arrangement by use of the crimper device in accordance with the present disclosure so as to obtain a leaflet folding arrangement of FIGS. 16 & 17;

FIGS. 19A-19B illustrate a side view and end view of the prosthetic heart valve that has been subjected to a non-limiting continuous progressive crimping process in accordance with the present disclosure;

FIGS. 20A-20B illustrate a side view and end view of the prosthetic heart valve that has been subjected to a non-limiting stepwise crimping process in accordance with the present disclosure;

FIG. 21 illustrates a non-limiting leaflet folding device that can optionally be used with the crimper device in accordance with the present disclosure to facilitate in obtaining a leaflet folding configuration during the crimping of the frame of a prosthetic heart valve so as to obtain a folded leaflet configuration after the frame has been fully crimped as illustrated in FIGS. 16, 17 and 22;

FIG. 22 illustrates the outflow end of a fully crimped frame of a prosthetic heart valve and wherein the arrows illustrate the counter-clockwise folding profile pattern of the leaflets so as to reduce the void spaced about the leaflets, and wherein the counter-clockwise folding profile pattern of the leaflets by the use of the crimping method in accordance with the present disclosure and the optional use of the leaflet folding device illustrated in FIG. 21;

FIG. 23 illustrates the outflow of a fully crimped frame of a prosthetic heart valve wherein the frame has been fully crimped by a prior art crimper and prior art crimping method, and wherein the leaflet configuration is disorganized which there by results in large void spaces between the leaflets and the crushing and the potential damaging of one or more of the leaflets;

FIG. 24 illustrates a non-limiting radially collapsible insert that can be used in a crimping method for crimping a prosthetic heart valve to obtain a leaflet folding arrangement that is the same or similar to the leaflet folding arrangement of FIGS. 16, 17 and 22.

FIG. 25 illustrates another non-limiting radially collapsible insert that includes one or more arms that can be used in a crimping method for crimping a prosthetic heart valve to obtain a leaflet folding arrangement that is the same or similar to the leaflet folding arrangement of FIGS. 16, 17 and 22.

FIG. 26 illustrates a non-limiting forming shaft that includes one or more arms that can be used in a crimping method for crimping a prosthetic heart valve to obtain a leaflet folding arrangement that is the same or similar to the leaflet folding arrangement of FIGS. 16, 17 and 22;

FIG. 27 illustrates a fold guide instrument that includes a plurality of prongs that can be used in a crimping method for crimping a prosthetic heart valve to obtain a leaflet folding arrangement that is the same or similar to the leaflet folding arrangement of FIGS. 16, 17 and 22; and

FIG. 28 is a table that illustrates the difference in the crimped outer diameter and the expanded outer diameter of the same prosthetic heart valve wherein one prosthetic heart valve was crimped by a traditional prior art crimping method and the other prosthetic heart valve was crimped by using one or more of the crimping methods in accordance with the present disclosure.

### DESCRIPTION OF NON-LIMITING EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

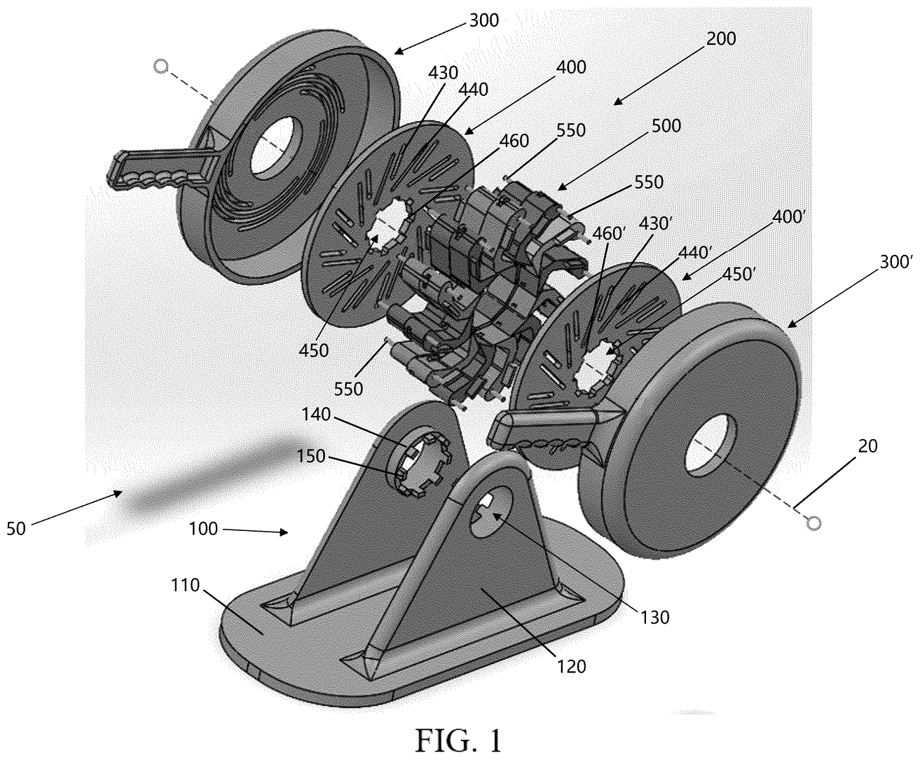

Referring now to the drawings wherein the showings are for the purpose of illustrating non-limiting embodiments of the disclosure only and not for the purpose of limiting same, FIG. 1-14B illustrate a non-limiting crimper device in accordance with the present disclosure.

Crimping a prosthetic heart valve is part of the process of transcatheter aortic valve implantation (TAVI). Crimping of the prosthetic heart valve is used to reduce the profile (or outer diameter) of the prosthetic heart valve, thereby making the prosthetic heart valve suitable for a minimally invasive catheter-based delivery. During the crimping of a prosthetic heart valve, the folding of one or more of the leaflets can be unorganized and result in damage to the leaflet and/or interfere with the crimping of the frame of the prosthetic heart valve. As the frame of the prosthetic heart valve is crimped and the outer diameter is reduced, such crimping causes the leaflets that are secured in the interior of the frame to begin folding. For traditional crimping processes wherein the most or the complete longitudinal length of the valve frame is simultaneously crimped, the folding arrangement of the leaflets is random due to various factors such as a) frame configuration, b) how the leaflets are mounted the frame, c) presence of an inner skirt in the interior of the frame, d) the leaflet configuration, e) the physical properties of the leaflets, f) the crimping parameters (e.g., speed of crimping, etc.), and g) the shape and/or orientation of the leaflets in the interior of the frame prior to the frame being crimped. Due to the many variables that affect the folding profile of the leaflets after the frame of the prosthetic heart valve is crimped, there is limited consistency from one crimped valve frame to another as to the folding profile of the leaflets for a particular valve frame. Also, after the frame has been crimped, it is difficult, if not impossible, to visually determine if one or more leaflets have been damaged during the crimping process. However, when the leaflets are not properly folded, the leaflets can be subjected to damage (e.g., leaflet tearing, damage between the connection of the leaflet and frame, damage to the connection between leaflets, creasing of the leaflet that adversely alters the shape of the leaflet when the frame is expanded at the treatment site, etc.—See FIG. 23), and the frame typically is prevented from being crimped to its smallest profile. Repeated applied crimping pressure to the frame to attempt to obtain smaller frame crimping profiles can result in a) no further crimping profile reduction, b) damage or further damage to one or more leaflets, c) damage to the inner and/or outer skirt, and/or d) damage to the frame.

Non-limiting implementations of the disclosed method, system and device provide a novel crimper device and methods of using the same that reduce the outer diameter or cross-sectional area of the frame of the prosthetic heart valve along a longitudinal length of the prosthetic heart valve. Although the crimping of a prosthetic heart will be described herein, it will be appreciated that the novel crimper device and methods of using the same can be used to crimp other types of medical devices that have a crimpable section or frame.

The crimper device in accordance with the present disclosure is configured to reduce the diameter or cross-sectional area of one or more portions of the frame either a) gradually (in a continuous manner) or b) stepwise (in a discreet manner) from i) the inflow side to the outflow side, or ii) from the outflow side to the inflow side, thereby causing a uniform and desirable folding pattern of the leaflets in the prosthetic heart valve, and which uniform leaflet folding pattern results in a reduced volume profile of the leaflets, which in turn results in a reduced outer diameter or cross-sectional area of the crimped prosthetic heart valve after crimping of the prosthetic heart valve is completed.

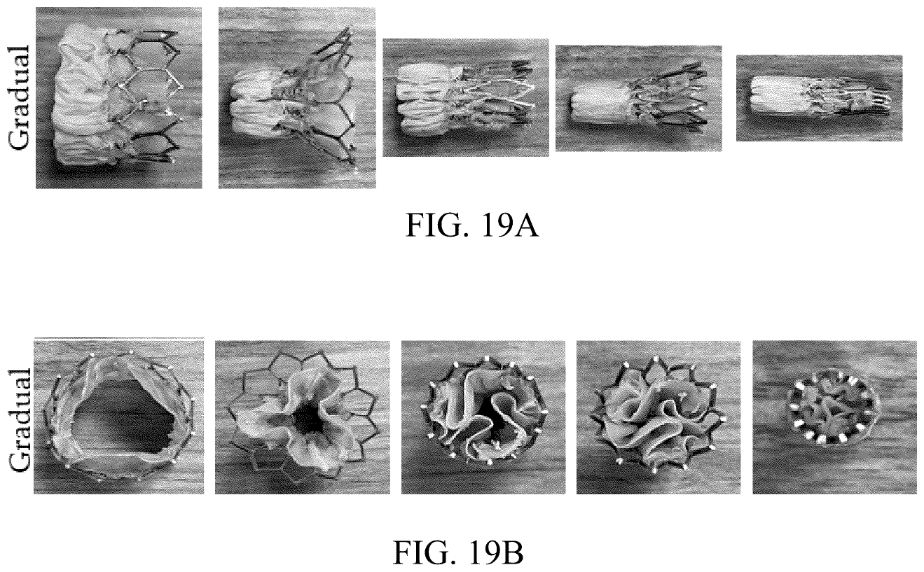

In either the continuous or stepwise crimping method, the inflow side portion or the outflow side portion of the prosthetic heart valve can be partially or fully crimped prior to crimping the other portions of the frame of the prosthetic heart valve. For most TAV and prosthetic heart valves, a portion of the inner and/or outer skirt are located at or near the inflow region and at least a portion of the leaflets are located above the inner and/or outer skirt at or near the outflow region as illustrated in FIGS. 19-21.

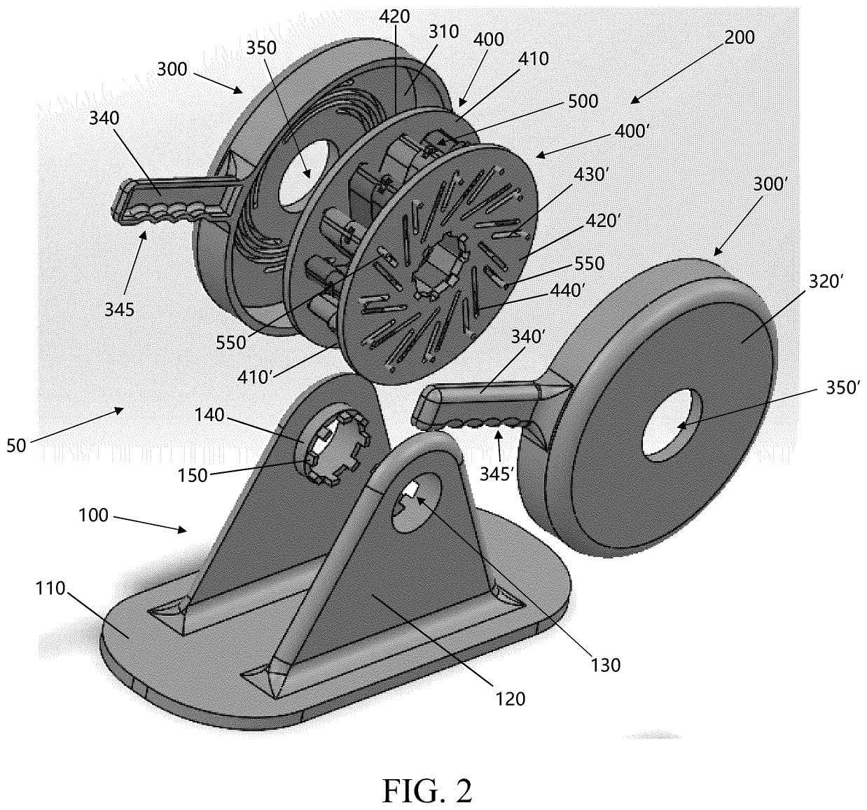

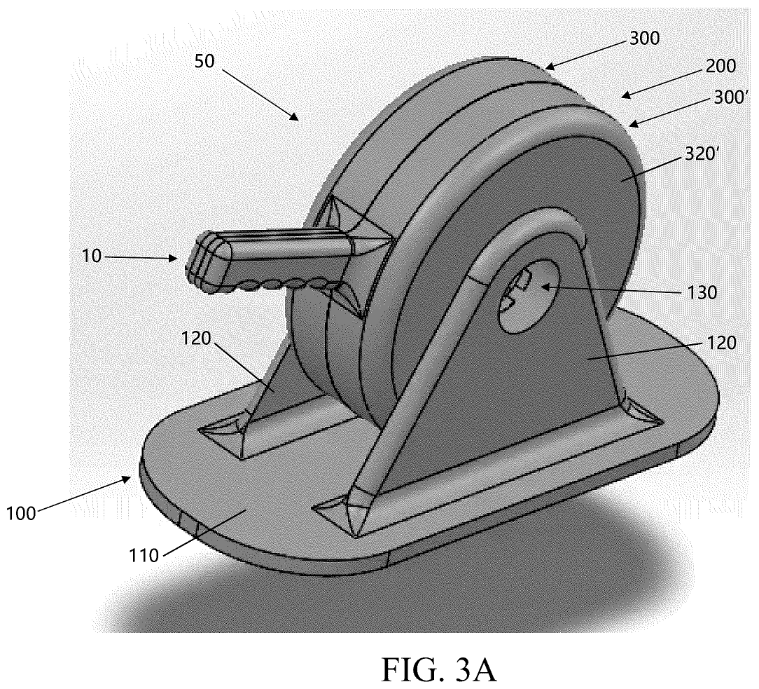

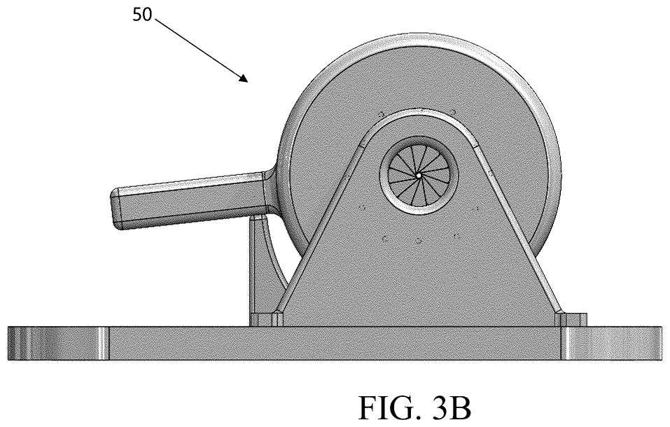

FIG. 1 depicts a non-limiting implementation of the crimper device in accordance with the present disclosure shown in an exploded configuration. FIG. 2 is a perspective view of the disclosed crimper device of FIG. 1 shown in a partially exploded configuration. FIG. 3A is a perspective view of the disclosed crimper device of FIG. 1 shown in a fully assembled configuration, and FIG. 3B is a side view of the disclosed crimper device of FIG. 1 shown in a fully assembled configuration.

With reference to FIGS. 1-3B, crimper device 50 includes a support assembly 100 and a crimper assembly 200. The crimper assembly 200 includes a first outer plate 300, a second outer plate 300′, a first inner plate 400, a second inner plate 400′, and a jaw assembly 500. In the present exemplary embodiment, crimper device 50 includes a single handle 10 that actuates the crimper assembly 200 around a central axis 20 of the crimper assembly 200. The single handle 10 is movable between an open position (the position of the handle when the size of opening 30 is the largest) and a closed position (the position of the handle when the size of opening 30 is the smallest). As can be appreciated, the crimper device 50 can include two or more handles.

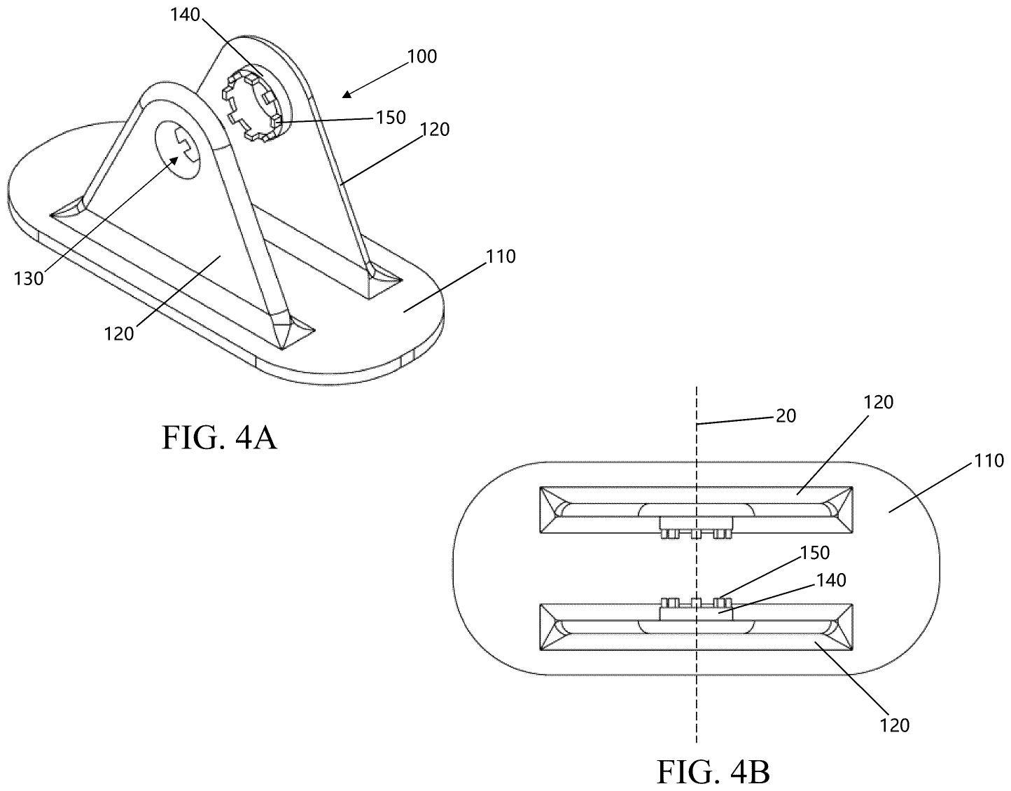

FIG. 4A provides a perspective view of the support assembly 100. FIG. 4B provides a top view of the support assembly 100. With reference to FIGS. 3A-3B and 4A-4B, support assembly 100 includes base 110 and two supports 120 that are connected to the base 110. Supports 120 each comprise coaxially aligned openings 130 having stops 140 with a plurality of protrusions 150. Stops 140 support the crimper assembly 200 at a predetermined height above base 110 so as to allow at least a portion of the crimper assembly 200 to move relative to the support assembly 100 during a crimping process. As can be appreciated, the configuration of the support assembly 100 is non-limiting, thus other types of support assemblies could be used to support the crimper assembly 200. The materials used for the support assembly 100 are also non-limiting. Generally, the support assembly is formed of a durable material (e.g., metal, plastic, ceramic material, composite material, etc.).

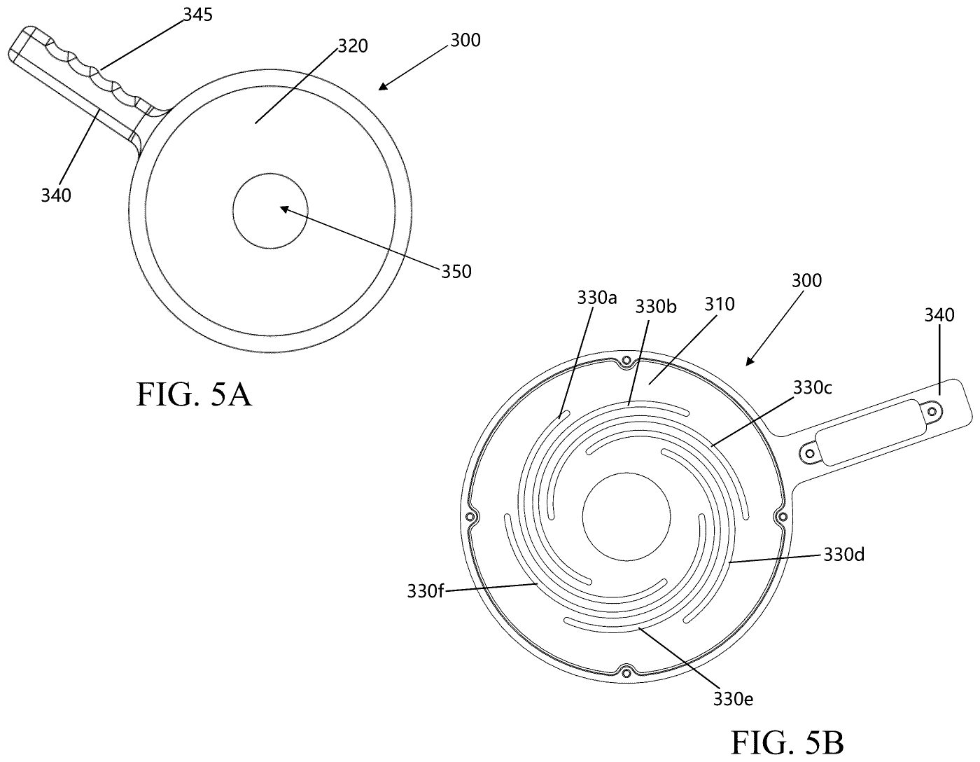

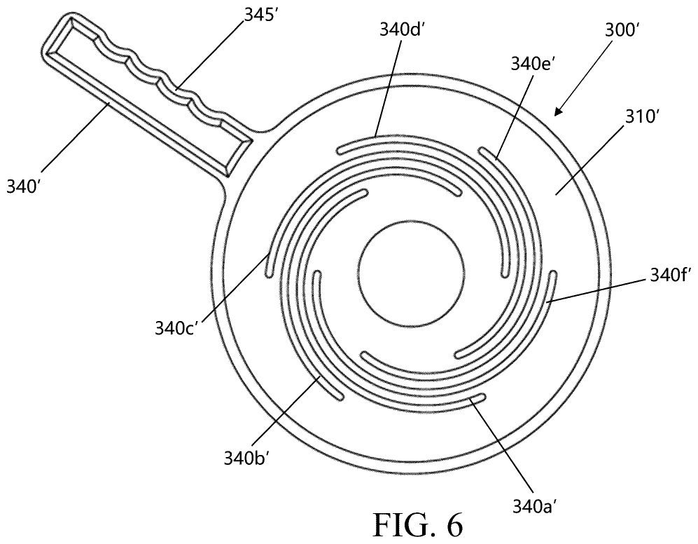

FIG. 5A provides an external view of the first outer plate 300, and FIG. 5B provides an internal view of the first outer plate 300. With reference to FIGS. 1, 3A, 3B and 5A-5B, first outer plate 300 includes an inner surface 310 and an outer surface 320. A plurality of tracks 330a, 330b, 330c, 330d, 330e, and 330f are disposed on the inner surface 310 of first outer plate 300. In the present exemplary embodiment, first outer plate 300 further comprise a handle member 340 having a plurality of optional finger grips 345. The finger grips 345, when used, facilitate in the grasping of the handle 10 by a user during the operation of the crimper device 50.

First outer plate 300 further defines an aperture 350 that is formed through inner surface 310 and outer surface 320. The aperture 350 that is coaxial with openings 130 of support assembly 100 when crimper device 50 is in a fully assembled configuration (see, for example, FIGS. 3A, 3B).

FIG. 6 provides an internal view of the second outer plate 300′. With reference to FIGS. 1, 3A, 3B, and 6, second outer plate 300′ includes an inner surface 310′ and an outer surface 320′. The inner surface 310′ includes a plurality of tracks 330a′, 330b′, 330c′, 330d′, 330e′, and 330f′. In the present exemplary embodiment, second outer plate 300′ further comprise a handle member 340′ having a plurality of optional finger grips 345′. The finger grips 345′, when used, facilitate in the grasping of the handle 10 by a user during the operation of the crimper device 50.

Second outer plate 300′ further defines an aperture 350′ formed through inner surface 310′ and outer surface 320′. The aperture 350′ is coaxial with openings 130 of support assembly 100 when crimper device 50 is in a fully assembled configuration (see, for example, FIGS. 3A, 3B).

In the present exemplary embodiment, second outer plate 300′ is a mirrored counterpart or close mirrored counterpart of first outer plate 300 such that tracks 330a′, 330b′, 330c′, 330d′, 330e′, and 330f of second outer plate 300′ correspond to tracks 330a, 330b, 330c, 330d, 330e, and 330f of first outer plate 300, and that handle member 345′ of second outer plate 300′ aligns with handle member 345 of first outer plate 300 when crimper device 50 is in a fully assembled configuration.

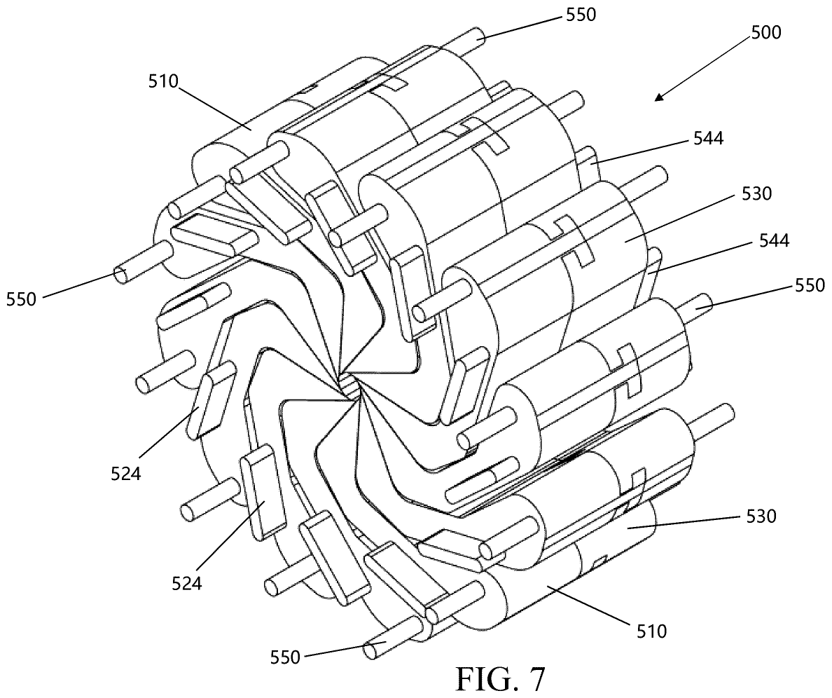

FIG. 7 is a perspective view of jaw assembly 500. The jaw assembly 500 includes a plurality of first jaws 510 and a plurality of second jaws 530 that are configured to form opening 30.

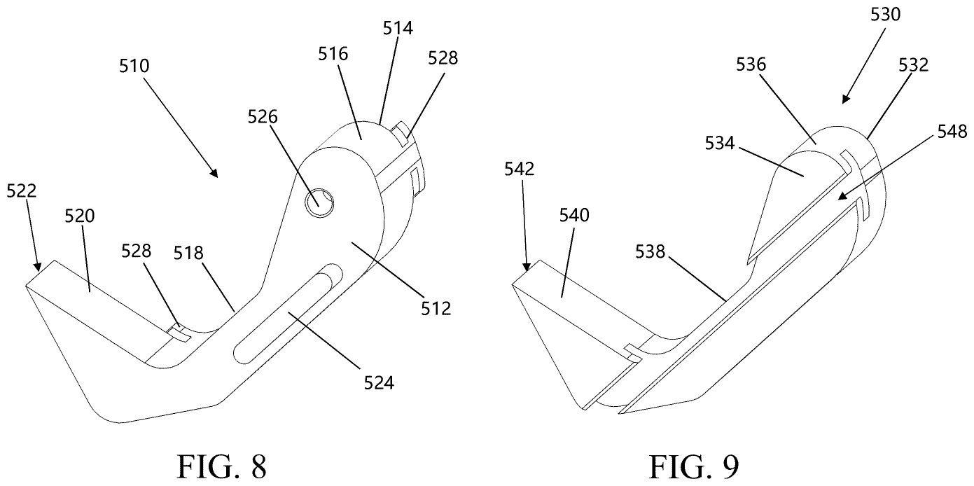

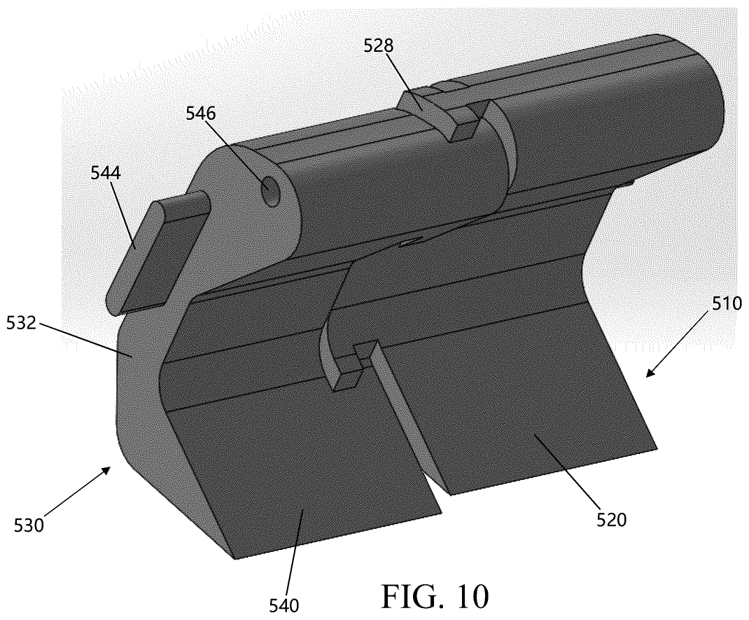

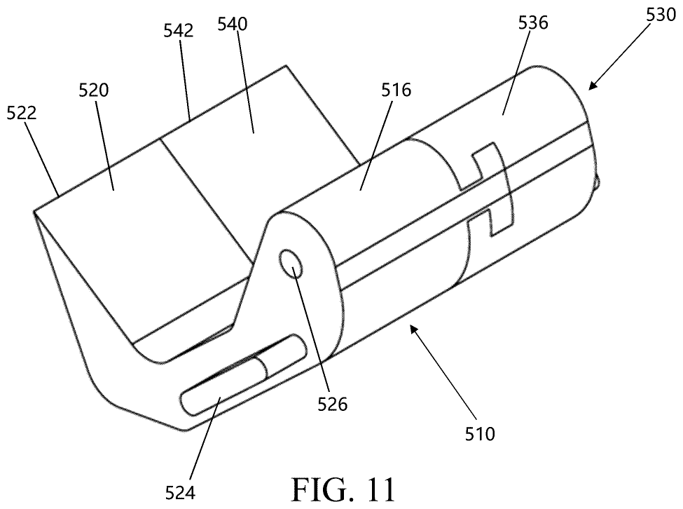

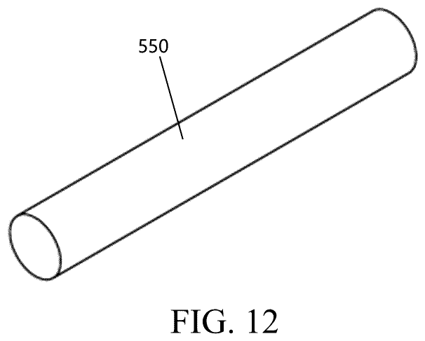

FIG. 8 is a perspective view of an exemplary one of first jaws 510 of the jaw assembly 500. FIG. 9 is a perspective view of an exemplary one or second jaws 530 of the jaw assembly 500. FIG. 10 is a perspective view of one of the first jaws 510 partially movably coupled to one of the second jaws 530. FIG. 11 is a perspective view of one of the first jaws 510 fully movably coupled to one of the second jaws 530. FIG. 12 is a perspective view of an exemplary pin 550 for use with the jaw assembly 500.

With reference again to FIGS. 7-12, the plurality of first jaws 510 each comprise an exterior surface 512, an interior surface 514, a head 516, a middle section 518, and a leg 520 that extends to a point 522. Exterior surface 512 includes a guide 524 and a hole 526. Hole 526 is formed in head 516. Coupling projections 528 are formed in the interior surface 514.

The plurality of second jaws 530 each comprise an exterior surface 532, an interior surface 534, a head 536, a middle section 538, and a leg 540 that extends to a point 542. Exterior surface 532 includes a guide 544 and a hole 546. Hole 524 is formed in head 536. Interior surface 534 defines coupling recess 548 that that is shaped and sized to slidably receive coupling projection 528 on first jaw 510 when first and second jaws 510, 530 are slidably coupled together. The configuration of coupling recess 548 and coupling projection 528 allow the first and second jaws 510, 530 to move independently from one another, but to also maintain the first and second jaws together as the first and second jaws 510, 530 are moved relative to one another. As can be appreciated, the configuration of the coupling recess 548 and coupling projection 528 are non-limiting. Any coupling arrangement can be used that allow the first and second jaws 510, 530 to move independently from one another, but to also maintain the first and second jaws together as the first and second jaws 510, 530 are moved relative to one another.

In the present exemplary embodiment, legs 520, 540 are displaced at a predetermined angle from middle sections 518, 538, respectively. As shown in FIGS. 7 and 9-10, coupling projections 528 of first jaws 510 engage coupling recesses 548 of second jaws such that the interior surface 514 of the first jaws 510 are flush with the interior surfaces 534 of the second jaws 530. Holes 526, 546 each receive a separate pin 550.

With reference to FIGS. 1-3B and 7, first inner plate 400 comprises an inner surface 410 and an outer surface 420. A plurality of pin channels 430 and a plurality of guide channels 440 are formed through and between the inner surface 410 and the outer surface 420. The length of the pin channels 430 is less than the length of the guide channels 440; however, this is not required.

Second inner plate 400′ comprises an inner surface 410′ and an outer surface 420′. A plurality of pin channels 430′ and a plurality of guide channels 440′ are formed through and between the inner surface 410′ and the outer surface 420′. The length of the pin channels 430′ is less than the length of the guide channels 440′; however, this is not required.

The first and second inner plates 400, 400′ each comprise a centrally formed aperture 450, 450′ having a plurality of cavities 460, 460′. The plurality of cavities 460, 460′ correspond to and receive a respective protrusion 150 of stops 140. Such an arrangement if configured to rigidly orient the first and second inner plates 400, 400′ to the two supports 120 such that when handle 10 is moved, the first and second inner plates 400, 400′ remain in a fixed position relative to the two supports 120 of the support assembly 100.

When crimper device 50 is in a fully assembled configuration, centrally formed apertures 450, 450′ are coaxial (a) with each other; (b) with apertures 350, 350′ of the first outer plate 300 and the second outer plate 300′; and (c) and with openings 130 of support assembly 100.

When crimper assembly 200 is assembled, pins 550 on first jaws 510 engage and pass through their respective pin channels 430 on first inner plate 400 in a slidable manner, and guides 524 on first jaws 510 engage their respective guide channels 440 on first inner plate 400 in a slidable manner. Guides 524 may or may not fully pass through the first inner plate. As can be appreciated, guide channels 440 may or may not fully extend between the inner surface 410 and outer surface 420 of first inner plate 400.

Likewise, when crimper assembly 200 is assembled, pins 550 on second jaws 530 engage their respective pin channels 430′ on second inner plate 400′ in a slidable manner, and guides 544 on second jaws 530 engage their respective guide channels 440′ on second inner plate 400′ in a slidable manner. Guides 544 may or may not fully pass through the second inner plate. As can be appreciated, guide channels 440′ may or may not fully extend between the inner surface 410′ and outer surface 420′ of second inner plate 400′.

As illustrated in FIGS. 1-2, pins 550 of the first and second jaws 510, 530 extend a predetermined distance past the outer surface 420 of first inner plate 400 and the outer surface 420′ of the second inner plate 400′, respectively, when the jaw assembly 500 is completely engaged with the inner plates 400, 400′. Pins 550 of first jaw 510 are configured to engage tracks 330a, 330b, 330c, 330d, 330e, and 330f of first outer plate 300 in a slidable manner; and pins 550 of second jaw 530 then engage tracks 330a′, 330b′, 330c′, 330d′, 330e′, and 330f′ of second outer plate 300′ in a slidable manner when the jaw assembly is fully assembled. As will be discussed in more detail below, when the handle is moved, the movement of the handle about the central axis 20 causes the first and second outer plates 300, 300′ to rotate about the central axis 20, which in turn causes pins 550 that are positioned in tracks 330a, 330b, 330c, 330d, 330e, and 330f of first outer plate 300 and pins that are positioned in tracks 330a′, 330b′, 330c′, 330d′, 330e′, and 330f′ of second outer plate 300′ to move within the respective tracks, which in turn causes the guides 524 on first jaws 510 to slide in respective guide channels 440 on first inner plate 400 and guides 544 on second jaws 530 to slide in respective guide channels 440′ on second inner plate 400′, which in turn causes the first and second jaws 510, 530 to move, which in turn causes the diameter or cross-sectional area of opening 30 to increase or decrease along a portion or all of the longitudinal length of opening 30.

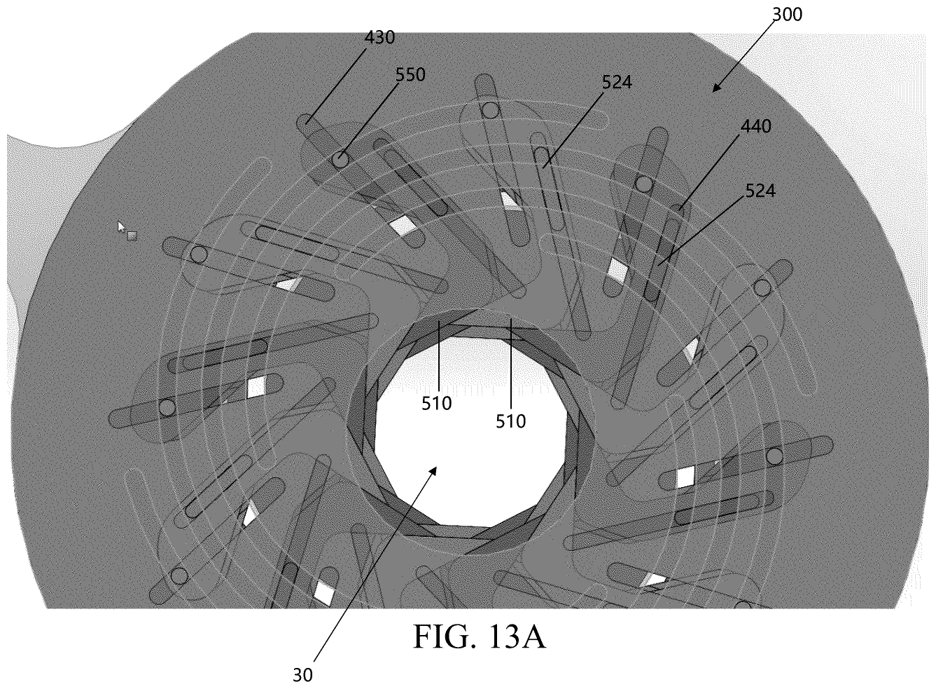

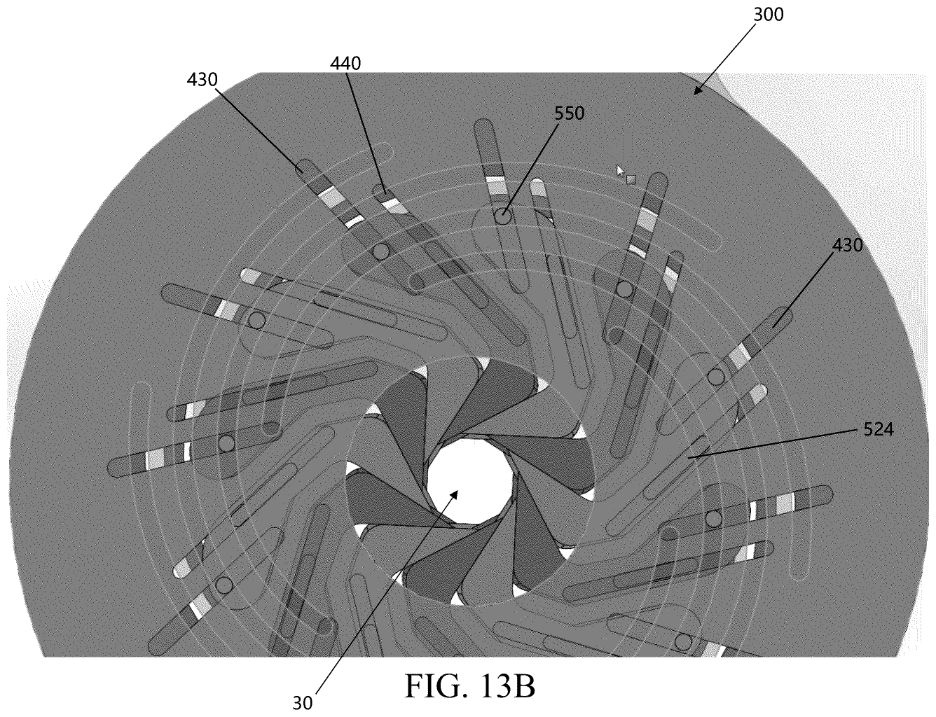

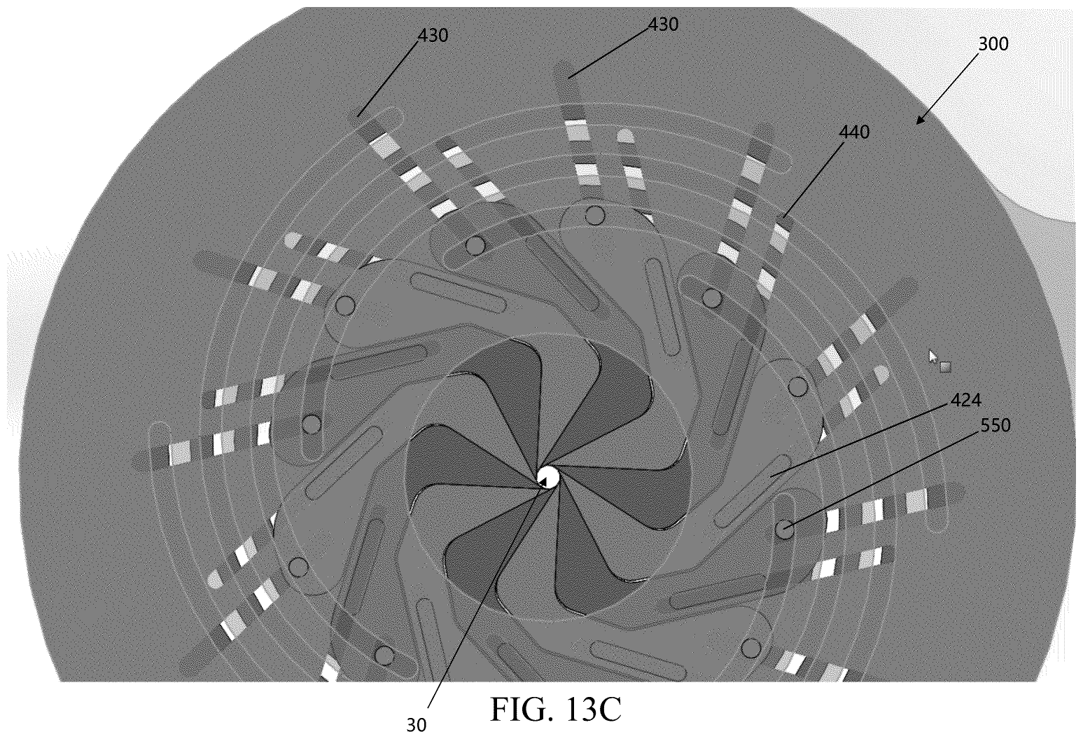

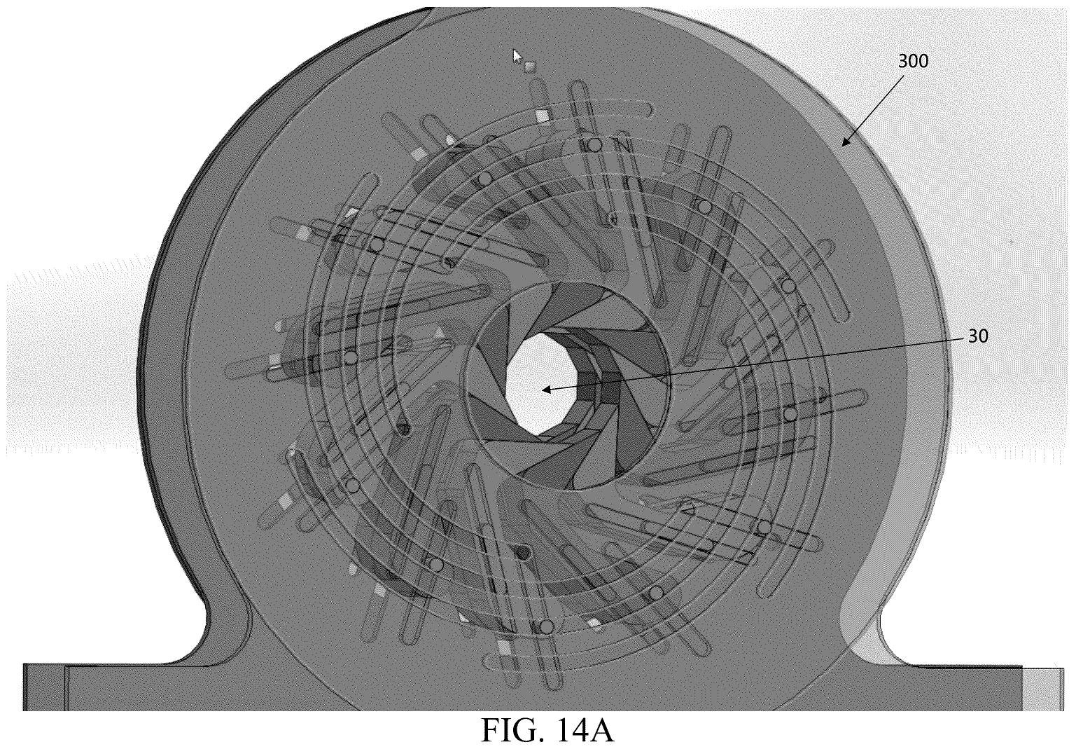

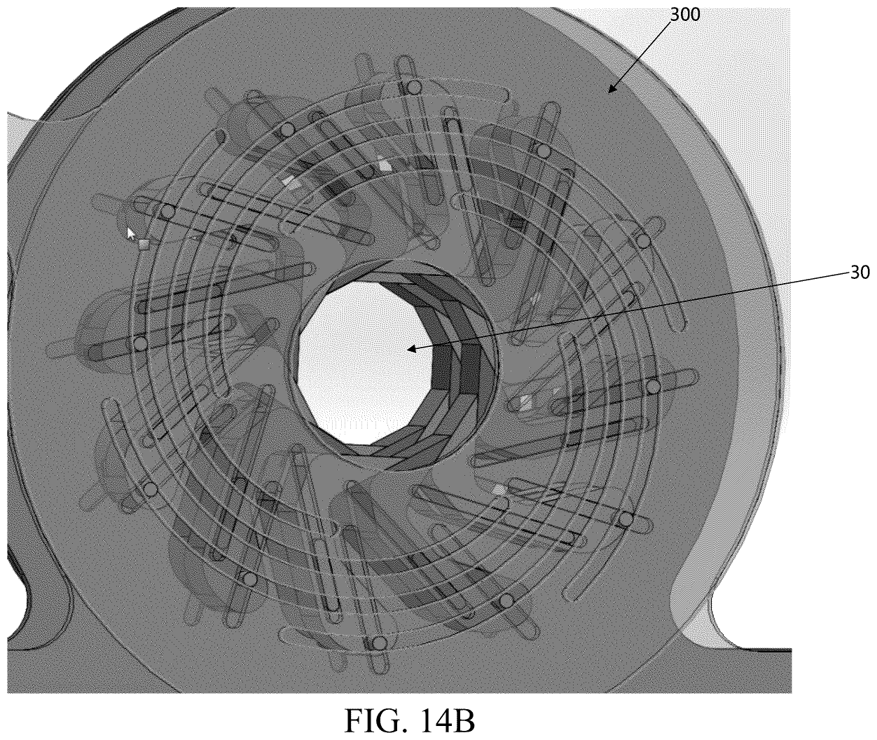

FIGS. 13A-13C provide perspective views of crimper device 50 and depict exemplary movement of the jaw assembly 500 from a partially opened position to a partially closed position. FIGS. 14A-14B provide perspective views of the crimper device 50 and depict exemplary movement of the jaw assembly 500 from a partially closed position to a partially opened position.

As the handle 10 is moved partially or fully around central axis 20: (a) pins 550 travel in their respective pin channels 430, 430′ of inner plates 400, 400′; (b) pins 550 travel in their respective tracks 330a-330f, 330a′-330f′ of outer plates 300, 300′; and (c) guides 524, 544 travel in their respective guide channels 440, 440′. When the handle 10 is moved towards the closed position (FIGS. 13A-13C), first jaws 510 and second jaws 530, sequentially, approach the center of the opening 30, thereby reducing the size of the opening 30 and applying a crimping force on a medical device that is located in opening 30, and wherein the crimping force is directed towards central axis 20. When handle 10 of the crimper device 50 is moved towards the open position (FIGS. 14A-14B), first jaws 510 and second jaws 530, sequentially, retreat from the center of opening 30, thereby increasing the size of the opening 30 and reducing or eliminating the crimping force that is applied to a medical device that is located in opening 30.

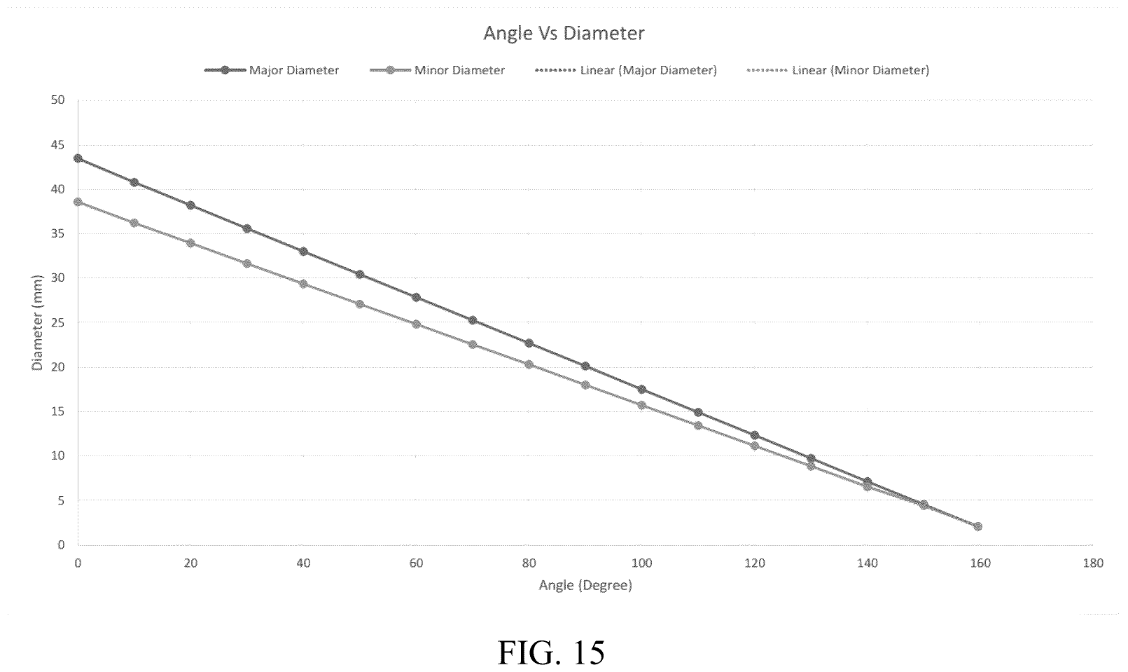

The sequential movement of the first jaws 510 and second jaws 530 can be configured to optionally provides a linear relationship between the rotational movement handle 20 relative to central axis 20 and the diameter of the opening 30 of jaw assembly 500. Such a linear relationship is illustrated by the graph in FIG. 15. As illustrated in FIG. 15, the crimper device 50 can be configured such that as the handle is moved from the fully open position (0°) to the fully closed or smallest diameter or cross-sectional area position for opening 30 (160°), the diameter of opening 30 that is formed by the first jaws 510 progressively decreases from 38 mm to 2.5 mm in a linear relationship to the rotational angle of handle 30, and the diameter of opening 30 that is formed by the second jaws 530 progressively decreases from 44 mm to 2.5 mm in a linear relationship to the rotational angle of handle 30. As represented in FIG. 15, the portion of opening 30 formed by first jaws 510 has a smaller diameter or cross-sectional area than the portion of opening 30 formed by second jaws 530 when the handle 10 is in the fully open position. As such, as handle 10 is moved from the fully opened position to the fully closed, the portion of the medical device that is located in the opening 30 formed by first jaws 510 encounters a crimping force from first jaws 510 prior to the portion of the medical device located in the portion of opening 30 that is formed by second jaws 530. However, as the handle 30 is continued to be moved to the fully closed or smallest diameter or cross-sectional area position for opening 30, the diameter of opening 30 formed by first jaws 510 and second jaws 530 begins to converge until the diameter of the opening 30 formed by the first and second jaws is the same when the handle 30 is moved to the fully closed or smallest diameter or cross-sectional area position for opening 30. As can be appreciated, the crimper assembly 200 can be configured to create other diameter or cross-sectional area relationships of opening 30 that are formed by the first and second jaws as the handle is moved from the fully opened position to the fully closed or smallest diameter or cross-sectional area position for opening 30.

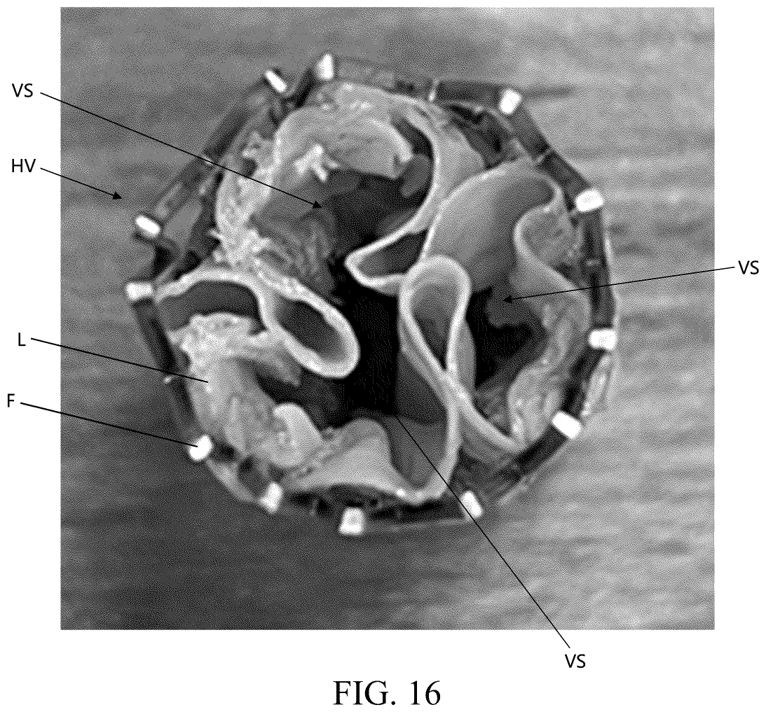

Referring now to FIG. 16, there is illustrated a rear-end view or outflow side end view of a crimped prosthetic heart valve that is has been partially crimped by the crimper device and crimping process as disclosed herein. As illustrated in FIG. 16, the leaflets are illustrated as beginning to be folded in an organized manner that reduces the number and volume of void spaces existing about the folded leaflets.

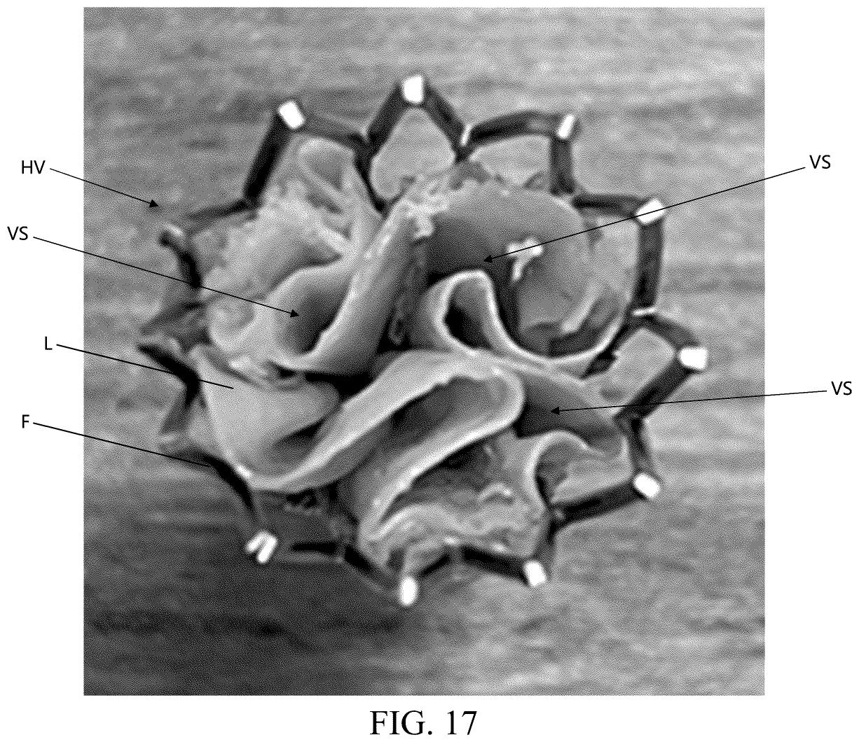

Referring now to FIG. 17, there is illustrated a rear-end view or outflow side end view of a crimped prosthetic heart valve showing the folding profile of leaflets in the fully crimped prosthetic heart valve that has been crimped by the crimper device and crimping process as disclosed herein. The prosthetic heart valve was crimped in such a way as to cause the leaflets to be folded in an organized manner that reduces the number and volume of void spaces about the folded leaflets, thereby enabling the frame of the prosthetic heart valve to be crimped to a smaller outer diameter.

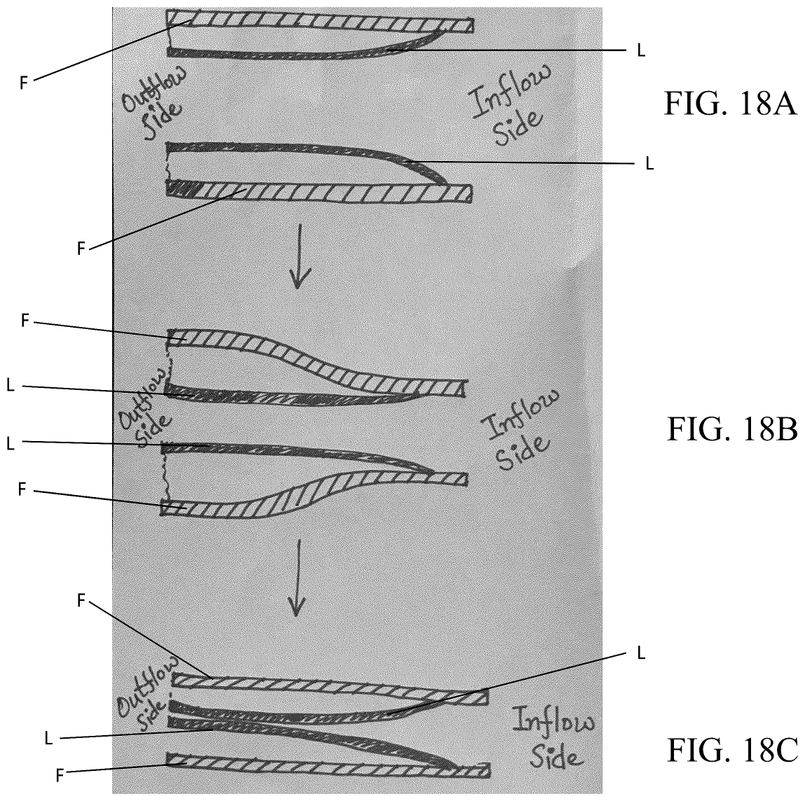

Referring now to FIGS. 18A-C, there is illustrated a three cross-sectional portions of a prosthetic heart valve along the longitudinal axis of the prosthetic heart valve wherein two leaflets are connected to the frame of the prosthetic heart valve. FIG. 18 illustrates the inflow side and outflow side of the prosthetic heart valve. The leaflets are illustrated as being connected to the frame at or near the inflow side of the prosthetic heart valve. FIG. 18A illustrates a cross-sectional portion of the prosthetic heart valve prior to the frame being subjected to a crimping force by the crimper assembly 200. For example, when the handle is in the fully open position and the prosthetic heart valve is positioned in opening 30, the diameter or cross-sectional area of opening 30 can be such that no crimping force is applied to the prosthetic heart valve.

FIG. 18B illustrates a cross-sectional portion of the prosthetic heart valve wherein the inflow region of the frame has been subjected to a crimping force to cause a reduction in diameter or cross-sectional area of the crimped frame; however, the outflow region of the frame has not yet been subjected to a crimping force, thus there has been no reduction in diameter or cross-sectional area of this region of the frame. For example, as the handle is moved from the fully opened position to the fully closed or smallest diameter or cross-sectional area position for opening 30 (160°), the first set of jaws 510 can be configured to first contacting and applying a crimping force to the inflow region of the prosthetic heart valve prior to the second set of jaws 530 contacting and applying a crimping force to the outflow region of the prosthetic heart valve.

FIG. 18C illustrates a cross-sectional portion of the prosthetic heart valve wherein both the inflow region and outflow regions of the frame has been subjected to a crimping force to cause a reduction in diameter or cross-sectional area of the crimped frame. For example, as the handle is continued to be moved to the fully closed or smallest diameter or cross-sectional area position for opening 30 (160°), the second set of jaws 530 eventually contact and apply a crimping force to the outflow region of the prosthetic heart valve while the first set of jaws 510 continue to apply a crimping force to the inflow region of the prosthetic heart valve. One non-limiting progressive crimping of the prosthetic heart valve is illustrated opening diameter v. handle rotation position as illustrated in FIG. 15.

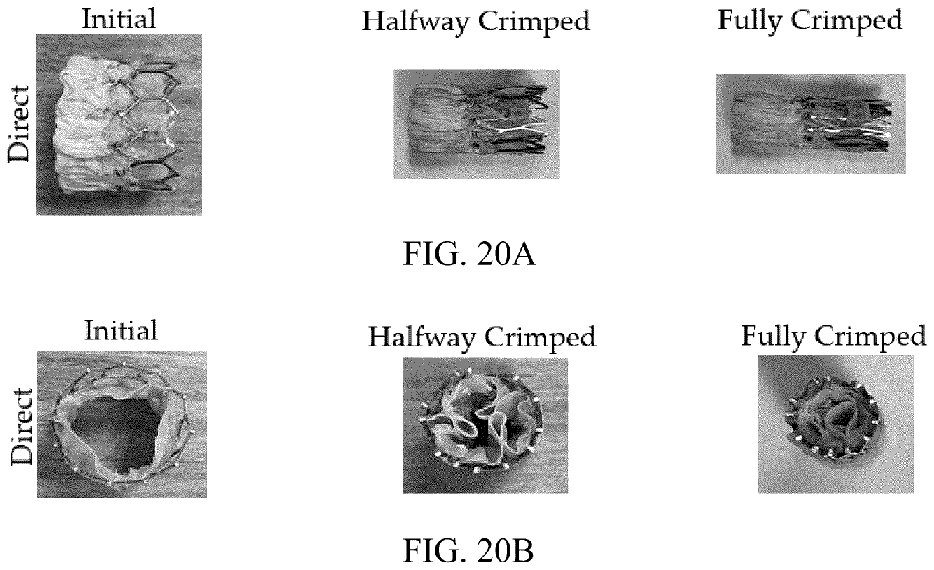

Referring now to FIGS. 19A, 19B, 20A, 20B, there are illustrated non-limiting crimping methods for a prosthetic heart valve using the disclosed crimper device, wherein: a) the prosthetic heart valve is progressively and continuously crimped along its longitudinal axis starting from the inflow end and progressing to and end at the outflow end (See FIGS. 19A-19B), or b) the inflow side portion of the prosthetic heart valve is first partially or fully crimped, and thereafter the outflow side portion of the prosthetic heart valve is partially or fully crimped (See FIGS. 20A-20B). Such a crimping method results in an organized leaflet profile of the crimped prosthetic heart valve that is the same or similar to the folded leaflet profile as illustrated in FIGS. 16, 17, 19B, 20B, and 22.

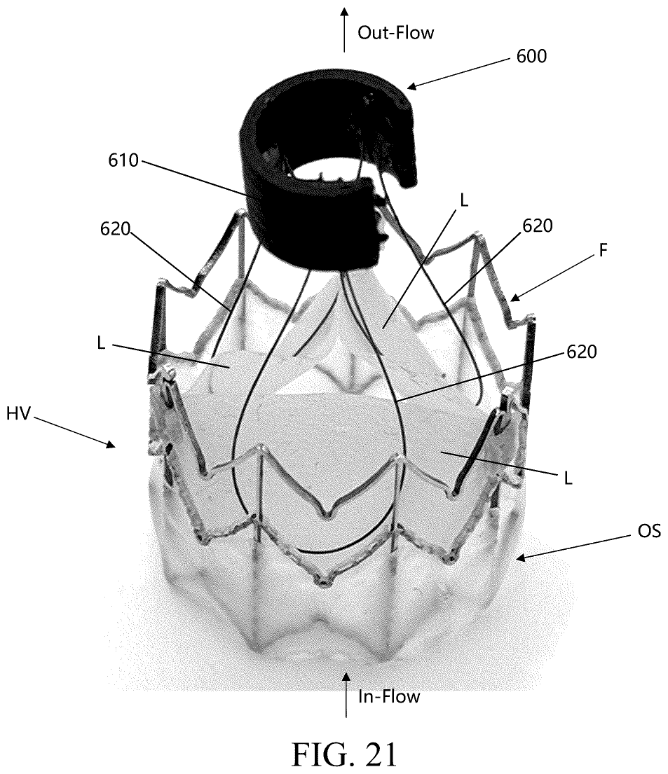

Referring now to FIG. 21, there is illustrated a prosthetic heart valve HV that includes a frame F, three leaflets L, and an inner skirt and an outer skirt OS. A leaflet folding device 600 is illustrated which can optionally be used with the crimper device to facilitate in obtaining a desired leaflet folding configuration during the crimping of the frame of a prosthetic heart valve. The leaflet folding device 600 includes a handle 610 and three leaflet engagement members 620 in the form of wire loops (e.g., metal wire loop, plastic wire loop, etc.) that are attached to and extend from the handle 610. FIG. 21 only illustrates a portion of the handle 610. Generally, the handle 610 is sized and shaped to such that it can be grasped by a user to enable the user to position the leaflet engagement members into a portion of the prosthetic heart valve to cause the bending of the one or more leaflets.

The leaflet engagement members 620 are attached to the distal end or distal end portion of the handle portion and are illustrated as extending radially outwardly from the central longitudinal axis of the handle portion. Generally, the leaflet engagement members have the same size, shape, and/or be formed of the same material; however, this is not required.

The leaflet engagement members can be formed of a flexible material that enables the leaflet engagement members to flex and/or bend a) when positioning the leaflet engagement members about one or more leaflets, and/or b) during the crimping of the frame of the prosthetic heart valve and while the leaflet engagement members are still engaged with the one or more leaflets during the crimping of the frame.

The leaflet folding device is configured to inwardly bend one or more or all of the end portions of the leaflets that are located at or near the outflow end of the prosthetic heart valve toward the central axis of the frame of the prosthetic heart valve. Such bending of the one or more leaflets by the leaflet folding device generally occurs a) prior to the initial crimping of the frame of the prosthetic heart valve, and/or b) during the crimping of the frame of the prosthetic heart valve. Generally, the leaflet folding device is removed from or disengaged from the one or more leaflets prior to the outflow end of the frame of the prosthetic heart valve being fully crimped to as to not interfere with the complete crimping of the frame of the prosthetic heart valve.

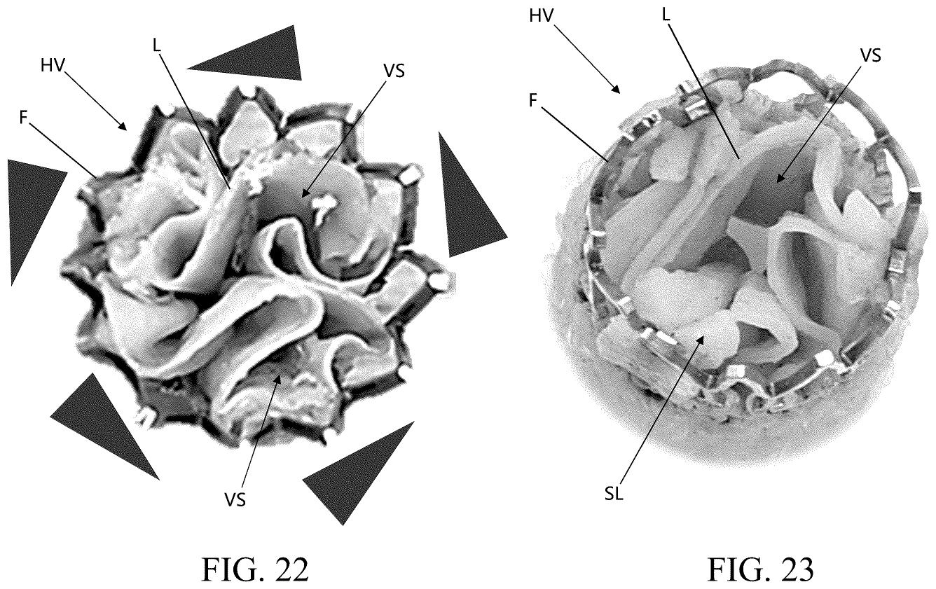

When the leaflet folding device 600 is optionally used, one non-limiting method of use is as follows: a) the leaflet folding device is moved along the longitudinal axis of the frame and toward the frame until the one or more leaflet engagement members engage the end or end portion of the one or more leaflets, b) thereafter, the leaflet folding device is continued to be moved along the longitudinal axis of the frame such that the end or end portion of the one or more leaflet engagement members move between the one or more leaflets and the inner surface of the frame, and c) thereafter, the leaflet folding device is continued to be moved along the longitudinal axis of the frame such that the angular orientation of the one or more leaflet engagement members relative to the central axis of the handle portion of the leaflet folding device causes the end and end portions of the leaflets to be bent toward the central axis of the frame. Generally, one or more leaflet engagement members are inserted only through a portion of the longitudinal length of the frame and are spaced from the region of the frame wherein the one or more leaflets are connected to the frame. Prior to and/or during the crimping of the frame, the handle portion of the leaflet folding device can be optionally rotated about the longitudinal axis of the frame so as to facilitate in the folding of the leaflets during the crimping of the frame. Such rotation is illustrated by the arrows in FIG. 22.

As illustrated in FIG. 23, when the leaflets of the prosthetic heart valve are not properly folded, the leaflets can be subjected to damage (e.g., leaflet tearing, damage between the connection of the leaflet and frame, damage to the connection between leaflets, creasing of the leaflet that adversely alters the shape of the leaflet when the frame is expanded at the treatment site, etc.), and the frame typically is prevented from being crimped to its smallest profile. Repeated applied crimping pressure to the frame to attempt to obtain smaller frame crimping profiles can result in a) no further crimping profile reduction, b) damage or further damage to one or more leaflets, c) damage to the inner and/or outer skirt, and/or d) damage to the frame. FIG. 23 illustrates a typically folding profile of leaflets F after the frame F of a prosthetic heart valve HV has been crimped by prior art crimping devices and methods. As illustrated in FIG. 23, the folding of the leaflets L is not organized as compared to the leaflet folding arrangement illustrated in FIG. 22. Also, there are significant void spaces VS about the folded leaflets L in the crimped prosthetic heart valve HV illustrated in FIG. 23. FIG. 22 illustrates a significantly less number and volume of void spaces VS between the organized folding of the leaflets L. FIG. 23 also illustrates some of the leaflets being smashed together (smashed Leaflets—SL) which can result in damage to the leaflets L. Such undesirable smashing of the leaflets L is illustrated in FIG. 22.

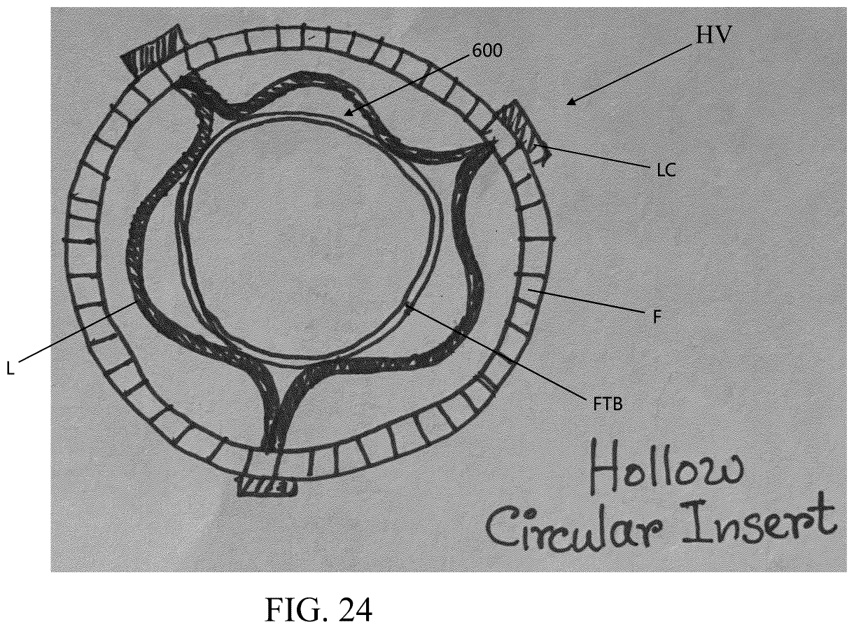

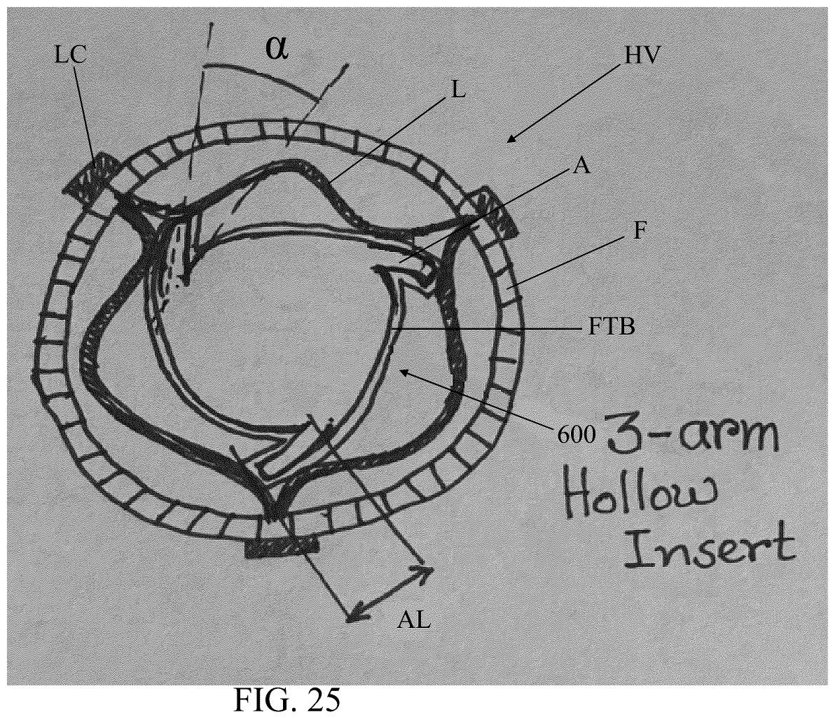

Referring now to FIGS. 24-25, there is illustrated a front end view or inflow side end view of a prior art prosthetic heart valve showing the use of two different leaflet folding devices 600 in the form of collapsible folding tube bodies FTB in accordance with the present disclosure that are used to obtain the leaflet folding arrangement that is the same or similar to the one illustrated in FIGS. 16, 17 and 22. The prosthetic heart valve HV includes a crimpable frame F, a plurality of leaflets L that are connected to the frame at one or more leaflet connection regions LC. FIG. 24 illustrates a folding tube body FTB that has a generally hollow cylindrical tube shape. FIG. 25 illustrates the folding tube body FTB having a similar shape to the folding tube body of FIG. 24, but also including three radially extending collapsible arms A. The angle α at which each arm initially extends from the outer surface of the body of the radially collapsible insert is 5-175° (and all values and ranges therebetween). The length of the arms AL is generally 3-10 mm (and all values and ranges therebetween). The collapsible folding tube bodies FTB are configured to be partially or fully inserted in the interior of the prosthetic heart valve prior to the crimping of the prosthetic heart valve. Once the collapsible folding tube body FTB is inserted in the interior of the prosthetic heart valve, the prosthetic heart valve is partially crimped which results in the partial or full collapse of the radially collapsible insert. Thereafter, the collapsible folding tube body FTB is removed from the prosthetic heart valve and the prosthetic heart valve is then again subjected to crimping until the prosthetic heart valve is fully crimped. The leaflet folding device can optionally be rotated and/or moved longitudinally along the longitudinal axis of the frame of prosthetic heart during the crimping of the prosthetic heart valve.

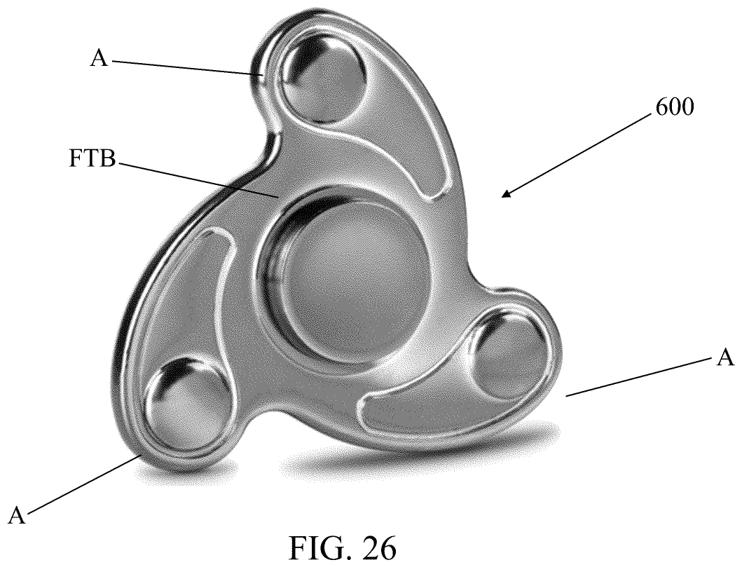

Referring now to FIG. 26, there is illustrated a leaflet folding device 600 in the form of a forming shaft FTB that includes three arms A that operates in a similar manner as the leaflet folding devices discussed above with reference to FIGS. 24 and 25, except that the body of the forming shaft is not collapsible. The arms on the forming shaft are used to obtain the leaflet folding arrangement that is the same or similar to the one illustrated in FIGS. 16, 17 and 22. The forming shaft is partially or fully inserted in the interior of the prosthetic heart valve prior to the crimping of the prosthetic heart valve. Once the forming shaft is inserted in the interior of the prosthetic heart valve such that the arms of the forming shaft are at least partially positioned in the interior of the prosthetic heart valve, the prosthetic heart valve is partially crimped. Thereafter, the forming shaft is removed from the prosthetic heart valve and the prosthetic heart valve is then again subjected to crimping until the prosthetic heart valve is fully crimped. The leaflet folding device can optionally be rotated and/or moved longitudinally along the longitudinal axis of the frame of prosthetic heart during the crimping of the prosthetic heart valve.

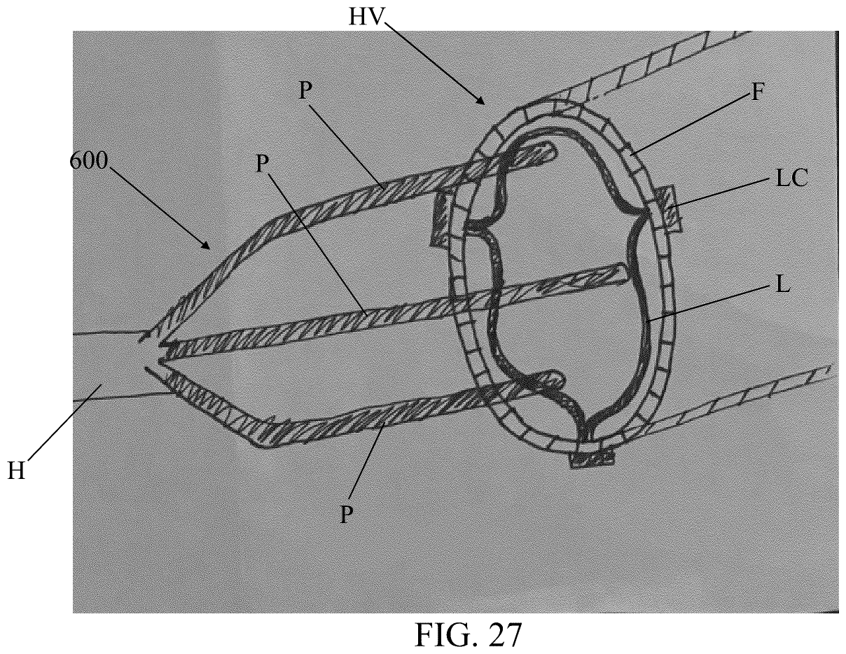

Referring now to FIG. 27, there is illustrated a front end or inflow side prospective view of a prosthetic heart valve HV showing the use of a leaflet folding device 600 in accordance with the present disclosure that is used to obtain the leaflet folding arrangement that is the same or similar to the one illustrated in FIGS. 16, 17 and 22. The leaflet folding device 600 includes a handle H and a plurality of prongs P that extend forwardly and radially outwardly from the central axis of the handle H. The prongs P are configured to be partially or fully inserted in the interior of the prosthetic heart valve prior to and/or during the crimping of the prosthetic heart valve HV. Once the prongs of the leaflet folding device 600 are inserted in the interior of the prosthetic heart valve, the prosthetic heart valve is partially crimped which results in the partial bending of the prongs. During the partial crimping of the prosthetic heart valve, the leaflet folding device 600 can be optionally rotated. Prior to the complete crimping of the prosthetic heart valve, the prongs of the leaflet folding device 600 are removed from the prosthetic heart valve and the prosthetic heart valve is then again subjected to crimping until the prosthetic heart valve is fully crimped.

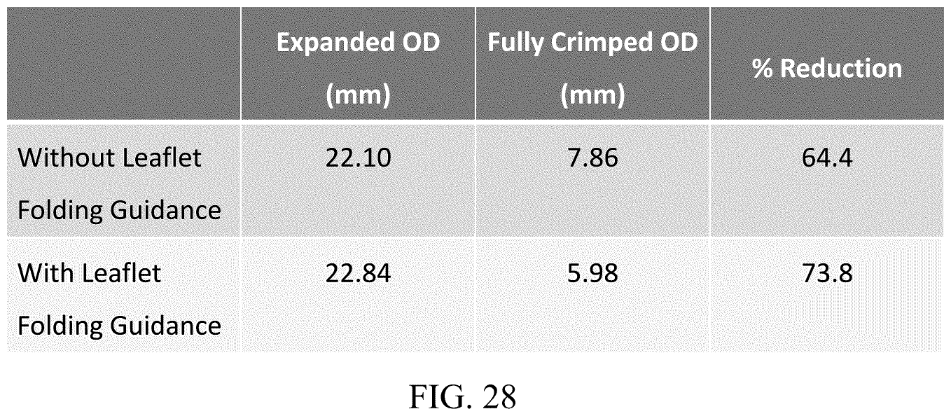

Referring now to FIG. 28, a table is shown comparing the outer crimped diameter of a prosthetic heart valve without using the crimping methods in accordance with the present disclosure to the crimped diameter of a prosthetic heart valve that has used one or more of the crimping methods in accordance with the present disclosure. The second column illustrates that a prior art prosthetic heart valve that is crimped without using the crimping methods in accordance with the present disclosure has a maximum fully crimped outer diameter of 7.86 mm and a 64.4% reduction in diameter compared to the diameter of the prosthetic heart valve prior to being crimped. The second column also illustrates the same prior art prosthetic heart valve that is crimped using one or more of the crimping methods in accordance with the present disclosure has a maximum fully crimped outer diameter of 5.98 mm and a 73.8% reduction in diameter compared to the diameter of the prosthetic heart valve prior to being crimped. As such, by controlling the folding profile of the leaflets by using one or more of the crimping methods in accordance with the present disclosure, a significantly smaller final crimped outer diameter of the prosthetic heart valve can be obtained.

The first column of FIG. 28 also compares the outer expanded diameter of a prosthetic heart valve that has been expanded from its crimped state wherein the prosthetic heart valve was crimped by a traditional prior art crimping method to the outer expanded diameter of a prosthetic heart valve that has been expanded from its crimped state wherein the prosthetic heart valve was crimped by using one or more of the crimping methods in accordance with the present disclosure. The prosthetic heart valve crimped by a traditional prior art crimping method is shown to obtain a smaller maximum outer diameter when expanded as compared to a prosthetic heart valve crimped by using one or more of the crimping methods in accordance with the present disclosure. It is believed that this increased outer diameter is due in part to the uniform folding of the leaflets that results from using one or more of the crimping methods in accordance with the present disclosure.

In operation, the crimper device can be configured to a) progressively crimp a frame of the prosthetic heart valve from one end to the other end, b) progressively crimp a frame of the prosthetic heart valve from one portion to another portion of the frame, and c) batch crimping of different portions of the frame at different times during the crimping process (e.g., crimping a front portion of the frame prior to crimping the rear portion of the frame, etc.). Such crimping process has been found to facilitate in the folding of the leaflets in a prosthetic heart valve in an organized manner so that the frame can be crimped to smaller crimped profiles and damage to the leaflets during the crimping process is reduced.

In one non-limiting embodiment, the crimper is configured to progressively crimp a frame of the prosthetic heart valve from one end to the other end (e.g., progressively crimp from the inflow end to the outflow end, etc.). In such an arrangement, the movement of the handle from the beginning position to the end position results in one end or end portion of the frame being initially subjected to the crimping process and subsequent portions of the frame being later subjected to the crimping process as the handle is continuously moved toward the end position. As illustrated in FIG. 15, the rate at which any portion of the frame is crimped as the handle moves from the beginning position to the end position can be or include a linear rate of crimping; however, such rate of crimping of one or more portions of the frame can have a non-linear rate of crimping.

When the handle is initially moved from the beginning position to the end position, the frame along 50-100% (and all values and ranges therebetween) of the longitudinal length of the frame can a) be immediately subjected or subjected within 0.0001-70% (and all values and ranges therebetween) distance of handle movement between the beginning position to the end position to a crimping force from the crimper device, or b) be subjected at different times to the crimping force form the crimper device. As such, during the crimping process, the crimping of the frame can result in a) all portions of the frame are crimped at a linear rate regarding the amount or percentage of crimping during 50-100% (and all values and ranges therebetween) of the crimping process of the frame, b) all portions of the frame are crimped at a same linear rate regarding the amount or percentage of crimping during 50-100% of the crimping process of the frame, and 50-100% (and all values and ranges therebetween) are crimped at the same linear rate of the amount or percentage of crimping during the crimping process, c) all portions of the frame are crimped at a linear rate of the amount or percentage of crimping during 50-100% of the crimping process of the frame, and 50-100% (and all values and ranges therebetween) are crimped at a different linear rate of the amount or percentage of crimping during the crimping process, d) 50-100% (and all values and ranges therebetween) the frame are crimped at different linear rate regarding the amount or percentage of crimping during 50-100% of the crimping process of the frame, or e) 1-100% (and all values and ranges therebetween) the frame are crimped at different linear rate regarding the amount or percentage of crimping during 1-100% (and all values and ranges therebetween) of the crimping process of the frame. As illustrated in FIG. 15, the crimper device can be configured to cause the first and second jaws 510, 530 to move as a linear rate such that opening 30 reduces in diameter at a linear or second order rate. As can be appreciated, during the movement of the handle between the beginning position to the end position, the user can move the handle at a constant or non-constant speed. For example, the user can move the handle at a generally constant speed from 10-100% (and all values and ranges therebetween) of the distance between the beginning position to the end position.

As can be appreciated, the manual movement of the handle can be replaced by a motorized movement mechanism. In such an arrangement, the motorized movement mechanism can be configured and/or controlled so as to a) move the handle at a generally constant speed from 1-100% (and all values and ranges therebetween) of the distance between the beginning position to the end position, b) move the handle at a generally non-constant speed from 1-100% (and all values and ranges therebetween) of the distance between the beginning position to the end position, or c) move the handle at some programmed speed profile as the handle moves between the beginning position to the end position.

In another non-limiting embodiment, the crimper device is configured to batch crimp the frame at different portions of the frame at different times during the crimping process. In such an arrangement, the crimper device can a) include two handles wherein movement of the first handle causes only first jaws 510 to move and the second handle causes only second jaws 530 to move, or b) includes a switching arrangement wherein when the single handle is initially moved, only first jaws 510 are cause to moved, and when the handle is moved back or near the beginning position, a switching arrangement is activated wherein when the handle is again moved to the end position only second jaws 530 are caused to move or both first and second jaws 510, 530 are caused to move. As can be appreciated, when a switching arrangement is used, the switching arrangement can be manual, mechanical or electro-mechanical switching arrangement. As also can be appreciated, the movement mechanism for the first and/or second jaws and the optional switching mechanism can be motorized movement mechanism that is similar to the motorized movement mechanism described above with regard to the single handle crimper device.

In one non-limiting configuration, the crimper device first applies a crimping force to a first end region of the frame that constitutes 1-80% (and all values and ranges therebetween) of the longitudinal length of the frame, and causes the first end region to be 10-100% (and all values and ranges therebetween) of fully crimped prior to applying a crimping force to a second end region of the frame that constitutes 20-99% (and all values and ranges therebetween) of the longitudinal length of the frame.

In another non-limiting configuration, the crimper device first applies a crimping force to a first end region of the frame that constitutes 20-60% of the longitudinal length of the frame, and causes the first end region to be 20-100% of fully crimped prior to applying a crimping force to a second end region of the frame that constitutes 40-80% of the longitudinal length of the frame.

In another non-limiting configuration, the crimper device first applies a crimping force to a first end region of the frame (e.g., inflow region of the prosthetic heart valve) that constitutes 30-60% of the longitudinal length of the frame, and causes the first end region to be 20-100% of fully crimped prior to applying a crimping force to a second end region of the frame (e.g., outflow region of the prosthetic heart valve) that constitutes 40-80% of the longitudinal length of the frame. In another non-limiting operation, when the crimper device applies a crimping force to a first end region of the frame, such first end region of the frame is the inflow end of the prosthetic heart valve. In another non-limiting operation, when the crimper device applies a crimping force to a first end region of the frame, such first end region of the frame is the outflow end of the prosthetic heart valve.

The example implementation of crimper device 50 shown in FIGS. 1-14B includes several structural features that assist the user of the crimper device in reducing the profile (or outer diameter) of the prosthetic heart valve, while minimizing the empty volume inside a prosthetic heart valve (especially in the leaflet area) and also inhibiting or preventing damage to the leaflets during the crimping process. It is to be appreciated that in accordance with another example implementation of the disclosed crimper device, the crimper device may also include one or more of: (a) at least a second crimping handle wherein the first crimping handle (when used) causes the crimper device to only crimp a first portion of the prosthetic heart valve and the second crimping handle (when used) causes the crimper device to crimp the remaining portion of the prosthetic heart valve that was not crimped when the first crimping handle was used; (b) a selection switch that includes a first and second setting wherein the first setting of the selection switch causes the single crimping handle (when used) to cause the crimper device to only crimp a first portion of the prosthetic heart valve and the second setting of the selection switch causes the single crimping handle (when used) to cause the crimper device to crimp the remaining portion of the prosthetic heart valve that was not crimped when the selection switch was at the first setting; (c) a limiting arrangement that limits the amount of crimping or reduction in outer diameter of the prosthetic device each time the crimping handle is used; and/or d) an automated arrangement that allows a user to select a crimping process (e.g., progressive crimping process, non-progressive crimping process, etc.) and parameters associated with the crimping process (e.g., crimping speed, linear or non-linear crimping forces applied to one or more portions of the prosthetic heart valve, etc.) that is specific for different types and sizes of prosthetic heart valves or frames of other devices that require crimping, and them performs the crimping process based on the selected crimping parameters.

As can be appreciated, the exemplary crimper device discussed herein can include more than two crimping handles, wherein each crimping handle is designed to cause (when the particular crimping handle is used) the crimper device to crimp a certain portion of the prosthetic heart valve. The two or more handles can be located on the same side of the crimper device, or one or more handles can be located on an opposite side of the crimper device from one or more other handles. As can also be appreciated, the exemplary crimper device discussed herein can include a selection switch that has two or more settings wherein each setting of the selection switch causes the crimper device (when the single crimping handle is used) to crimp a certain portion of the prosthetic heart valve. As can also be appreciated, the crimper device can include two or more crimping handles that are interconnected, and wherein actuation or use of the first crimping handle causes actuation of its associated set of actuating jaws to partially or fully crimp a portion of the frame of the prosthetic heart valve and, after the use of the first crimping handle, the second crimping handle can be used to partially or fully crimp another portion of the frame of the prosthetic heart valve. If the crimper device includes further crimping handles, such crimping handles can be sequentially allowed to crimp other portions of the frame in a similar fashion as described above with regard to the first and second crimping handles.

As can also be appreciated, the exemplary crimper device discussed herein can include a plurality of the sets of actuating jaws that are interconnected and controlled by a single crimping handle. During use, the single crimping handle causes sequential actuation of the different sets of actuating jaws to progressively crimp the prosthetic heart valve from one end to the other end (e.g., from the inflow side to the outflow side (or vice versa)). As can also be appreciated, the crimper device can include multiple crimping handles, a selection switch that includes two or more settings, and/or interconnected and progressively activated jaws to combine the operating arrangements as discussed above. As can also be appreciated, the crimper device can include a crimping limiter to limit the amount that the crimper device crimps the prosthetic heart valve each time the crimper device is used, or each or a certain crimping handle is used. The crimping limiter can include two or more settings. In one non-limiting arrangement, one or more settings of the crimping limiter limits the crimper device to crimp the prosthetic heart valve 10-95% (and all values and ranges therebetween) of its final crimped outer diameter. In another non-limiting arrangement, at least one of the settings of the crimping limiting allows the crimper device to achieve full crimping of at least a portion of the prosthetic heart valve. As can be appreciated, the one or more handles on the crimper device, the selection switch, progressive operation of the jaws, and/or crimping limiter can be replaced by a circuit board and/or other types of circuitries to partially or fully automate the crimping process of the crimper device and/or allow a user to input one or more settings to crimp the prosthetic heart valve by the crimper device.

Furthermore, the crimper devices discussed herein can be used in various non-limiting methods to minimize the empty volume inside a prosthetic heart valve and inhibit or prevent damage to the leaflets during the crimping process. In one non-limiting example, the inflow side portion of the prosthetic heart valve is crimped 10-100% (and all values and ranges therebetween) of the full or total crimped profile prior to crimping the outflow side portion of the prosthetic heart valve. In another non-limiting example, the outflow side portion of the prosthetic heart valve is crimped 10-100% (and all values and ranges therebetween) of the full or total crimped profile prior to crimping the inflow side portion of the prosthetic heart valve. When one side portion is not fully crimped prior to the crimping of the other side portion, the side portion to first be crimped can be crimped 10-99% (and all values and ranges therebetween) of its full crimped profile prior to the other side portion being crimped 10-100% (and all values and ranges therebetween) of its full crimped profile. As can be appreciated, one side portion can be first partially crimped, and then the other side portion can be partially crimped, then the side that was first partially crimped can be further crimped, and then the outer side portion can be further crimped, and this process can be repeated until the prosthetic heart valve is fully crimped.

In accordance with another non-limiting aspect of the present disclosure, the inflow side portion of the prosthetic heart valve is first substantially (e.g., 80-99.99% fully crimped and all values and ranges therebetween) or fully crimped, and then subsequently the outflow side portion of the prosthetic heart valve is substantially (e.g., 80-99.99% fully crimped and all values and ranges therebetween) or fully crimped. This crimping process in accordance with the present disclosure is a stepwise crimping process. As can be appreciated, the stepwise crimping process can alternately include the step of first substantially (e.g., 80-99.99% fully crimped and all values and ranges therebetween) or fully crimping the outflow side portion of the prosthetic heart valve, and then subsequently substantially (e.g., 80-99.99% fully crimped and all values and ranges therebetween) or fully crimping the inflow side portion of the prosthetic heart valve. After the prosthetic heart valve is initially subjected to the progressive continuous crimping process, the prosthetic heart valve can optionally be subjected to one or more additional crimping process to ensure that the smallest outer diameter of the prosthetic heart valve is obtained. Such optional additional crimping processes include a) a traditional prior art crimping process wherein the complete prosthetic heart valve is subjected to crimping forces, b) a stepwise crimping process wherein one portion of the prosthetic heart valve is subjected to crimping forces and then other portions of the prosthetic heart valve are subjected to crimping forces, and/or c) a progressive continuous crimping process wherein the crimping of the prosthetic heart valve starts at the inflow end or the outflow end and the crimping continuously progresses along the longitudinal length of the prosthetic heart valve to the opposite end of the prosthetic heart valve.

In accordance with another non-limiting aspect of the present disclosure, the prosthetic heart valve is gradually and continuously crimped from the inflow side and then continuing along the longitudinal length of the prosthetic heart valve to the outflow side to progressively crimp the prosthetic heart valve along its longitudinal length until the prosthetic heart valve is partially crimped (e.g., 50-99.99% fully crimped and all values and ranges therebetween) or fully crimped. As can be appreciated, the progressive continuous crimping process can alternatively include the step of gradually crimping the prosthetic heart valve starting from the outflow side and continuing to the inflow side until the prosthetic heart valve is fully crimped. After the prosthetic heart valve is initially subjected to the progressive continuous crimping process, the prosthetic heart valve can optionally be subjected to one or more additional crimping process to ensure that smallest outer diameter of the prosthetic heart valve is obtain. Such optional additional crimping processes include a) a traditional prior art crimping process wherein the complete prosthetic heart valve is subjected to crimping forces, b) a stepwise crimping process wherein one portion of the prosthetic heart valve is subjected to crimping forces and then other portions of the prosthetic heart valve are subjected to crimping forces, and/or c) a progressive continuous crimping process wherein the crimping of the prosthetic heart valve starts at the inflow end or the outflow end and the crimping continuously progresses along the longitudinal length of the prosthetic heart valve to the opposite end of the prosthetic heart valve.

In accordance with another non-limiting aspect of the present disclosure, the inflow side portion of the prosthetic heart valve is first partially crimped (inflow side portion crimped 10-80% of full crimping and all values and ranges therebetween), and then subsequently the outflow side portion of the prosthetic heart valve is partially crimped (outflow side portion crimped 10-80% of full crimping and all values and ranges therebetween), and then either: (a) crimping the inflow side portion until it is fully crimped, and then further crimping the outflow side portion until it is fully crimped; (b) further crimping the outflow side portion, and then further crimpling the inflow side portion until it is fully crimped; (c) performing a traditional prior art crimping process wherein the complete prosthetic heart valve is subjected to crimping forces to fully crimp the prosthetic heart valve; (d) performing a progressive continuous crimping process is used wherein the crimping of the prosthetic heart valve starts at the inflow end and processes to the outflow end; or (e) performing a progressive continuous crimping process wherein the crimping of the prosthetic heart valve starts at the outflow end and processes to the inflow end.

Conversely, and in accordance with still another non-limiting aspect of the present disclosure, the user may first partially crimp the outflow side portion of the prosthetic heart valve (outflow side portion crimped 10-80% of full crimping and all values and ranges therebetween), and then subsequently partially crimp the inflow side portion (inflow side portion crimped 10-80% of full crimping and all values and ranges therebetween) of the prosthetic heart valve, and then either: (a) further crimp the inflow side portion until it is fully crimped, and then further crimp the outflow side portion until it is fully crimped; (b) further crimp the outflow side portion until it is fully crimped, and then further crimp the inflow side portion until it is fully crimped; (c) perform a traditional prior art crimping process wherein the complete prosthetic heart valve is subjected to crimping forces to fully crimp the prosthetic heart valve; (d) perform a progressive continuous crimping process wherein the crimping of the prosthetic heart valve starts at the inflow end and processes to the outflow end; or (e) perform a progressive continuous crimping process wherein the crimping of the prosthetic heart valve starts at the outflow end and processes to the inflow end.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall there between. The disclosure has been described with reference to the certain embodiments. These and other modifications of the disclosure will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, Applicant does not intend any of the appended claims or claim elements to invoke 35 U.S.C. 112(f) unless the words “means for” or “step for” are explicitly used in the particular claim.

1. A method for reducing a profile of a frame of a medical device; said method comprising:
providing said medical device that includes a crimpable or plastically deformable portion;
providing a crimping device; said crimping device includes a crimping assembly that has a device opening; said device opening is configured to receive a portion or all of said crimpable or plastically deformable portion of said medical device; said device opening is configured to both reduce and expand in diameter or cross-sectional area during operation of said crimping device;
inserting at least a portion of said crimpable or plastically deformable portion of said medical device into said device opening; and
operating said medical device to cause at least a portion of said device opening to reduce in diameter or cross-sectional area to thereby exert a crimping force initially on only a first portion of said crimpable or plastically deformable portion of said medical device which causes said first portion of said crimpable or plastically deformable portion of said medical device to reduce in diameter or cross-sectional area, and thereafter continuing to use said crimping device to subsequently exert a crimping force on a second portion of said crimpable or plastically deformable portion of said medical device which causes said second portion of said crimpable or plastically deformable portion of said medical device to reduce in diameter or cross-sectional area; said second portion of said crimpable or plastically deformable portion of said medical device is spaced along a longitudinal axis of said medical device from said first portion of said crimpable or plastically deformable portion of said medical device; said first portion of said crimpable or plastically deformable portion of said medical device constitutes to 0.001-75% of a longitudinal length of said crimpable or plastically deformable portion of said medical device; said second portion of said crimpable or plastically deformable portion of said medical device constitutes to 0.001-75% of a longitudinal length of said crimpable or plastically deformable portion of said medical device.

2. The method as defined in claim 1, wherein said crimping device is configured to continue to exert said crimping force on both said first and second portions of said crimpable or plastically deformable portion of said medical device after initially exerting said crimping force on said second portion so as to further simultaneously reduce said diameter or cross-sectional area of both said first and second portions of said crimpable or plastically deformable portion of said medical device.

3. The method as defined in claim 2, wherein said crimping device is configured to exert said crimping force on said first portion until said diameter or cross-sectional area of said first portion of said crimpable or plastically deformable portion of said medical device is reduced by at least 1% prior to said crimping device initially exerting said crimping force on said second portion of said crimpable or plastically deformable portion of said medical device.

4. The method as defined in claim 2, wherein said crimping device is configured to exert said crimping force on said first portion until said diameter or cross-sectional area of said first portion of said crimpable or plastically deformable portion of said medical device is reduced by at least 5% prior to said crimping device initially exerting said crimping force on said second portion of said crimpable or plastically deformable portion of said medical device.

5. The method as defined in claim 2, wherein said crimping device is configured to exert said crimping force on said first portion until said diameter or cross-sectional area of said first portion of said crimpable or plastically deformable portion of said medical device is reduced by at least 25% prior to said crimping device initially exerting said crimping force on said second portion of said crimpable or plastically deformable portion of said medical device.

6. The method as defined in claim 2, wherein said crimping device is configured to exert said crimping force on said first portion until said diameter or cross-sectional area of said first portion of said crimpable or plastically deformable portion of said medical device is reduced by at least 50% prior to said crimping device initially exerting said crimping force on said second portion of said crimpable or plastically deformable portion of said medical device.

7. The method as defined in claim 1, wherein said first and second portions of said crimpable or plastically deformable portion of said medical device are both simultaneously positioned in said device opening during reduction of said diameter or cross-sectional area of said first and second portions of said crimpable or plastically deformable portion of said medical device by said crimper device; said first and second portions of said crimpable or plastically deformable portion of said medical device constitute 100% of a longitudinal length of said crimpable or plastically deformable portion of said medical device.

8. The method as defined in claim 1, wherein said first portion of said crimpable or plastically deformable portion of said medical device includes an inflow end portion of said medical device.

9. (canceled)

10. The method as defined in claim 1, wherein said second portion of said crimpable or plastically deformable portion of said medical device includes an outflow end portion of said medical device.

11. (canceled)

12. The method as defined in claim 1, wherein said crimping assembly includes a first set of jaws and a second set of jaws; said first set of jaws includes first and second jaws; said second set of jaws includes first and second jaws; said first jaw of said first and second set of jaws are positioned adjacent to one another and are slidably connected to one another to enable said first jaw of said first and second set of jaws to independently move with respect to one another while being connected to one another; said second jaw of said first and second set of jaws are positioned adjacent to one another and are slidably connected to one another to enable said second jaw of said first and second set of jaws to independently move with respect to one another while being connected to one another.

13. The method as defined in claim 12, wherein said crimper device includes a support assembly that supports said crimper assembly; said crimper assembly further includes a first outer plate, a second outer plate, a first inner plate, and a second inner plate; said first inner plate engages each of said jaws of said first set of jaws of said crimping assembly; each of said jaws of said first set of jaws are independently movable relative to said first inner plate; said second inner plate engages each of said jaws of said second set of jaws of said crimping assembly; each of said jaws of said second set of jaws are independently movable relative to said second inner plate; said first inner plate is positioned on one side of said crimping assembly and said second inner plate is positioned on an opposite side of said crimping assembly.

14. The method as defined in claim 13, wherein said first and second inner plates are non-rotatably connected to said support assembly; said first and second outer plates are rotatably connected to said support assembly; said first outer plate engages each of said jaws of said first set of jaws of said crimping assembly; said second outer plate engages each of said jaws of said second set of jaws of said crimping assembly.

15. The method as defined in claim 13, wherein each of said first and second jaws of said first set of jaws includes a pin or flange that at least partially forms an engagement with both said first inner plate and said first outer plate; each of said first and second jaws of said second set of jaws includes a pin or flange that at least partially forms an engagement with both said second inner plate and said second outer plate.

16. The method as defined in claim 15, wherein each of said first and second inner plates include a plurality of slots; each of said slots of said first inner plate receives a portion of said pin or flange from one of said jaws of said first set of jaws; each of said pin or flange of each of said jaws of said first set of jaws is movable within said respective slot when said first outer plate is moved relative to said support assembly; movement of said pin or flange of each of said jaws of said first set of jaws within said respective slot of said first inner plate results in said first set of jaws causing a portion of said device opening to change in diameter or cross-sectional area; each of said pin or flange of each of said jaws of said second set of jaws is movable within said respective slot when said second outer plate is moved relative to said support assembly; movement of said pin or flange of each of said jaws of said second set of jaws within said respective slot of said second inner plate results in said second set of jaws causing a portion of said device opening to change in diameter or cross-sectional area.

17. The method as defined in claim 16, wherein said device opening has a longitudinal length along a longitudinal axis of said device opening; said first set of jaws causing a first portion of said device opening to change in diameter or cross-sectional area; said second set of jaws causing a second portion of said device opening to change in diameter or cross-sectional area; said first portion of said device opening constitutes 0.1-70% of said longitudinal length of said device opening; said second portion of said device opening constitutes 0.1-70% of said longitudinal length of said device opening.

18. The method as defined in claim 17, wherein said first and section portions of said device opening are positioned adjacent to one another or are spaced from one another along said longitudinal axis of said device opening.

19. The method as defined in claim 1, wherein said medical device includes one or more leaflets that are connected to said crimpable or plastically deformable portion of said medical device.

20. The method as defined in claim 19, wherein said medical device is a prosthetic heart valve; said crimpable or plastically deformable portion of said medical device at least partially forms a frame of said prosthetic heart valve; said one or more leaflets are connected to said frame.

21. The method as defined in claim 19, further includes the step of providing a leaflet folding device that is configured to facilitate in folding said one or more of said leaflet during said step of applying said crimping force on said crimpable or plastically deformable portion of said medical device.

22. The method as defined in claim 21, further including the steps of:
at least partially inserting said leaflet folding device inside at least a portion of said prosthetic heart valve prior to fully crimping said prosthetic heart valve;
applying said crimping force to said prosthetic heart valve while said leaflet folding device is at least partially inserted in said prosthetic heart valve; and
removing said leaflet folding device from said prosthetic heart valve prior to said prosthetic heart valve being fully crimped.

23. The method as defined in claim 21, further including the step of a) moving said leaflet folding device along said longitudinal length of said frame during said step of applying said crimping force to said prosthetic heart valve while said leaflet folding device is at least partially inserted in said prosthetic heart valve, and/or b) rotating said leaflet folding device in said frame during said step of applying said crimping force to said prosthetic heart valve while said leaflet folding device is at least partially inserted in said prosthetic heart valve.

24. The method as defined in claim 19, further including the step of causing at least a portion of one or more of said leaflets to be bent toward a central longitudinal axis of said crimpable or plastically deformable portion of said medical device a) prior to applying said crimping force on said crimpable or plastically deformable portion of said medical device, and/or b) while applying said crimping force to said crimping of said crimpable or plastically deformable portion of said medical device.

25. The method as defined in claim 19, further includes the step of applying a rotational force about a central longitudinal axis of said crimpable or plastically deformable portion to one or more of said leaflets a) prior to applying said crimping force on said crimpable or plastically deformable portion of said medical device, and/or b) while applying said crimping force to said crimping of said crimpable or plastically deformable portion of said medical device.

26. The method as defined in claim 21, further including the step of positioning at least a portion of said leaflet folding device between said one or more leaflets and said crimpable or plastically deformable portion of said medical device to facilitate in a) bending at least a portion of one or more of said leaflets toward said central longitudinal axis of said crimpable or plastically deformable portion, and/or b) rotating at least a portion of one or more of said leaflets about said central longitudinal axis of said crimpable or plastically deformable portion.

27. The method as defined in claim 21, wherein said leaflet folding device includes a handle portion and one or more leaflet engagement members that are attached to and extend from said handle portion; said one or more leaflet engagement members are at least partially formed of a flexible material that enables said one or more leaflet engagement members to flex and/or bend a) when positioning said one or more leaflet engagement members at last partially about one or more leaflets, and/or b) as said crimpable or plastically deformable portion of said medical device reduces in diameter or cross-sectional area as said crimping force is applied on said crimpable or plastically deformable portion of said medical device.

28. The method as defined in claim 27, further including the step of positioning a portion of one or more of said leaflet engagement members between a portion of one or more of said leaflets and said crimpable or plastically deformable portion of said medical device to cause at least a portion of one or more of said leaflets to bend towards said central longitudinal axis of said crimpable or plastically deformable portion of said medical device.

29. A crimper device that is configured to crimp a crimpable or plastically deformable portion of a medical device; said crimping device includes a crimping assembly that has a device opening; said device opening is configured to receive a portion or all of the crimpable or plastically deformable portion of the medical device; said device opening is configured to both reduce and expand in diameter or cross-sectional area of the crimpable or plastically deformable portion of the medical device during operation of said crimping device; a first portion of said device opening is configured to a) reduce in diameter or cross-sectional area at a different rate as compared to a second portion of said device opening during said operation of said crimping device, and/or b) begin reducing in diameter or cross-sectional area at a different time as compared to a second portion of said device opening during said operation of said crimping device; said device opening has a longitudinal length along a longitudinal axis of said device opening; said first portion of said device opening constitutes 0.1-70% of said longitudinal length of said device opening; said second portion of said device opening constitutes 0.1-70% of said longitudinal length of said device opening.

30. The crimper device as defined in claim 29, wherein said first and section portions of said device opening are positioned adjacent to one another or are spaced from one another along said longitudinal axis of said device opening.

31. The crimper device as defined in claim 29, wherein said crimping assembly includes a first set of jaws and a second set of jaws; said first set of jaws includes first and second jaws; said second set of jaws includes first and second jaws; said first jaw of said first and second set of jaws are positioned adjacent to one another and are slidably connected to one another to enable said first jaw of said first and second set of jaws to independently move with respect to one another while being connected to one another; said second jaw of said first and second set of jaws are positioned adjacent to one another and are slidably connected to one another to enable said second jaw of said first and second set of jaws to independently move with respect to one another while being connected to one another.

32. The crimper device as defined in claim 31, wherein said crimper device includes a support assembly that supports said crimper assembly; said crimper assembly further includes a first outer plate, a second outer plate, a first inner plate, and a second inner plate; said first inner plate engages each of said jaws of said first set of jaws of said crimping assembly; each of said jaws of said first set of jaws are independently movable relative to said first inner plate; said second inner plate engages each of said jaws of said second set of jaws of said crimping assembly; each of said jaws of said second set of jaws are independently movable relative to said second inner plate; said first inner plate is positioned on one side of said crimping assembly and said second inner plate is positioned on an opposite side of said crimping assembly.

33. The crimper device as defined in claim 32, wherein said first and second inner plates are non-rotatably connected to said support assembly; said first and second outer plates are rotatably connected to said support assembly; said first outer plate engages each of said jaws of said first set of jaws of said crimping assembly; said second outer plate engages each of said jaws of said second set of jaws of said crimping assembly.

34. The crimper device as defined claim 32, wherein each of said first and second jaws of said first set of jaws includes a pin or flange that at least partially forms an engagement with both said first inner plate and said first outer plate; each of said first and second jaws of said second set of jaws includes a pin or flange that at least partially forms an engagement with both said second inner plate and said second outer plate.

35. The crimper device as defined in claim 32, wherein each of said first and second inner plates include a plurality of slots; each of said slots of said first inner plate receives at portion of said pin or flange from one of said jaws of said first set of jaws; each of said pin or flange of each of said jaws of said first set of jaws is movable within said respective slot when said first outer plate is moved relative to said support assembly; movement of said pin or flange of each of said jaws of said first set of jaws within said respective slot of said first inner plate results in said first set of jaws causing a portion of said device opening to change in diameter or cross-sectional area; each of said pin or flange of each of said jaws of said second set of jaws is movable within said respective slot when said second outer plate is moved relative to said support assembly; movement of said pin or flange of each of said jaws of said second set of jaws within said respective slot of said second inner plate results in said second set of jaws causing a portion of said device opening to change in diameter or cross-sectional area.
